(19) United States
(12) Patent Application Publication
(10) Pub. No.: US 2022/0288461 A1
(43) Pub. Date: Sep. 15, 2022

(54) MATHEMATICAL MODELING FOR PREDICTION OF OCCUPATIONAL TASK READINESS AND ENHANCEMENT OF INCENTIVES FOR REHABILITATION INTO OCCUPATIONAL TASK READINESS

(71) Applicant: ROM TECHNOLOGIES, INC., Brookfield, CT (US)

(72) Inventors: John Ashley, San Francisco, CA (US); Steven Mason, Las Vegas, NV (US)

(73) Assignee: ROM TECHNOLOGIES, INC., Brookfield, CT (US)

(21) Appl. No.: 17/827,095

(22) Filed: May 27, 2022

Related U.S. Application Data
(63) Continuation-in-part of application No. 17/739,906, filed on May 9, 2022.
Application No. 17/739,906 is a continuation of application No. 17/150,938, filed on Jan. 15, 2021, now Pat. No. 11,325,005.
Application No. 17/150,938 is a continuation-in-part of application No. 17/021,895, filed on Sep. 15, 2020, now Pat. No. 11,071,597.
(60) Provisional application No. 62/910,232, filed on Oct. 3, 2019.
(60) Provisional application No. 63/066,488, filed on Aug. 17, 2020.
(60) Provisional application No. 63/194,772, filed on May 28, 2021.

(51) Int. Cl. A63B 24/00; A63B 21/005; G16H 20/30; G06N 20/00; G16H 10/60

(52) U.S. Cl. CPC A63B 24/0075; A63B 24/0062; A63B 21/0058; G16H 20/30; G06N 20/00; G16H 10/60; A63B 2022/0094

(57) ABSTRACT

A method includes receiving first data pertaining to a first user using a treatment apparatus to perform a treatment plan, wherein the first data comprises an attribute of the first user and an occupational task associated with the first user; receiving second data pertaining to a second user, wherein the second data comprises an attribute of the second user and an occupational task associated with the second user; determining whether an attribute and occupational task of the second user matches an attribute and occupational task of the first user; and responsive to determining that the attribute and the occupational task of the second user matches the attribute and occupational task of the first user, predicting, via an artificial intelligence engine, an estimate of when the second user performing the treatment plan would be capable of performing the occupational task associated with the second user.

### CROSS-REFERENCES TO RELATED APPLICATIONS

This application is a continuation-in-part of U.S. patent application Ser. No. 17/739,906 filed May 9, 2022, titled “Systems and Methods for Using Machine Learning to Control an Electromechanical Device Used for Prehabilitation, Rehabilitation, and/or Exercise”, which is a continuation of U.S. patent application Ser. No. 17/150,938, filed Jan. 15, 2021, titled “Systems and Methods for Using Machine Learning to Control an Electromechanical Device Used for Prehabilitation, Rehabilitation, and/or Exercise”, which is a continuation-in-part of U.S. patent application Ser. No. 17/021,895, filed Sep. 15, 2020, titled “Telemedicine for Orthopedic Treatment”, which claims priority to and the benefit of U.S. Provisional Patent Application Ser. No. 62/910,232, filed Oct. 3, 2019, titled “Telemedicine for Orthopedic Treatment”, the entire disclosures of which are hereby incorporated by reference for all purposes. U.S. patent application Ser. No. 17/150,938, also claims priority to and the benefit of U.S. Provisional Patent Application Ser. No. 63/066,488, filed Aug. 17, 2020, titled “Systems and Methods for Using Machine Learning to Control an Electromechanical Device Used for Prehabilitation and/or Exercise”, the entire disclosure of which is hereby incorporated by reference for all purposes.

This application also claims priority to and the benefit of U.S. Provisional Patent Application Ser. No. 63/194,772, filed May 28, 2021, titled “Mathematical Modeling for Prediction of Occupational Task Readiness and Enhancement of Incentives for Rehabilitation into Occupational Task Readiness”, the entire disclosure of which is hereby incorporated by reference for all purposes.

This application is also a continuation of International Application No. PCT/US22/31023, filed May 26, 2022, titled “System and Method for Generating Treatment Plans to Enhance Patient Recovery Based on Specific Occupations”, the entire disclosure of which is hereby incorporated by reference for all purposes.

### BACKGROUND

Remote medical assistance, or telemedicine, may aid a patient in performing various aspects of a rehabilitation regimen for a body part. The patient may use a patient interface in communication with a healthcare professional interface for receiving the remote medical assistance via audio and/or audiovisual communications.

### SUMMARY

In one embodiment, a method may include receiving first data pertaining to a first user using a treatment apparatus to perform a treatment plan, wherein the first data comprises at least one attribute of the first user and at least one attribute of an occupational task associated with the first user; receiving second data pertaining to a second user, wherein the second data comprises at least one attribute of the second user and at least one attribute of an occupational task associated with the second user; determining whether at least one attribute of the second user matches with at least one attribute of the first user, and whether at least one attribute of the occupational task associated with the second user matches with at least one attribute of the occupational task associated with the first user; and responsive to determining that at least one attribute of the second user matches with at least one attribute of the first user and that at least one attribute of the occupational task associated with the second user matches with at least one attribute of the occupational task associated with the first user, predicting, via an artificial intelligence engine, an estimate of when the second user performing the treatment plan would be capable of performing the occupational task associated with the second user.

In one embodiment, a system includes a memory device storing instructions; a processing device communicatively coupled to the memory device, and the processing device executes the instructions to: receive first data pertaining to a first user using a treatment apparatus to perform a treatment plan, wherein the first data comprises at least one attribute of the first user and at least one attribute of an occupational task associated with the first user; receive second data pertaining to a second user, wherein the second data comprises at least one attribute of the second user and at least one attribute of an occupational task associated with the second user; determine whether at least one attribute of the second user matches with at least one attribute of the first user, and whether at least one attribute of the occupational task associated with the second user matches with at least one attribute of the occupational task associated with the first user; and responsive to determining that at least one attribute of the second user matches with at least one attribute of the first user and that at least one attribute of the occupational task associated with the second user matches with at least one attribute of the occupational task associated with the first user, predict, via an artificial intelligence engine, an estimate of when the second user performing the treatment plan would be capable of performing the occupational task associated with the second user.

In one embodiment, a tangible, non-transitory computer-readable medium stores instructions that, when executed, cause a processing device to perform any of the methods, operations, or steps described herein.

### BRIEF DESCRIPTION OF THE DRAWINGS

For a detailed description of example embodiments, reference will now be made to the accompanying drawings in which:

FIG. 1 shows a block diagram of an embodiment of a computer implemented system for managing a treatment plan according to the present disclosure;

FIG. 2 shows a perspective view of an embodiment of a treatment apparatus according to the present disclosure;

FIG. 3 shows a perspective view of a pedal of the treatment apparatus of FIG. 2 according to the present disclosure;

FIG. 4 shows a perspective view of a person using the treatment apparatus of FIG. 2 according to the present disclosure;

FIG. 5 shows an example embodiment of an overview display of a healthcare professional interface according to the present disclosure;

FIG. 6 shows an example block diagram of training the machine learning model to output an estimate of when a patient performing the treatment plan would be capable of performing the occupational task, based on data pertaining to the patient, data pertaining to an occupational task associated with the patient, and data pertaining to a treatment plan;

FIG. 7 shows an embodiment of an overview display of the healthcare professional interface presenting recommended treatment plans and excluded treatment plans in real-time during a telemedicine session according to the present disclosure;

FIG. 8 shows an embodiment of the overview display of the healthcare professional interface presenting, in real-time during a telemedicine session, recommended treatment plans that have changed as a result of patient data changing according to the present disclosure;

FIG. 9 shows an example embodiment of a method for selecting, based on assigning a patient to a cohort, a treatment plan for the patient and controlling, based on the treatment plan, a treatment apparatus according to the present disclosure;

FIG. 10 shows an example embodiment of a method for presenting, during a telemedicine session, the recommended treatment plan to a healthcare professional according to the present disclosure; and

FIG. 11 shows an example computer system according to the present disclosure.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or a direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

A “treatment plan” may include one or more treatment protocols, and each treatment protocol includes one or more treatment sessions. Each treatment session comprises several session periods, with each session period including a particular exercise for treating the body part of the patient. For example, a treatment plan for post-operative rehabilitation after a knee surgery may include an initial treatment protocol with twice daily stretching sessions for the first 3 days after surgery and a more intensive treatment protocol with active exercise sessions performed 4 times per day starting 4 days after surgery. A treatment plan may also include information pertaining to a medical procedure to perform on the patient, a treatment protocol for the patient using a treatment apparatus, a diet regimen for the patient, a medication regimen for the patient, a sleep regimen for the patient, additional regimens, or some combination thereof.

The terms telemedicine, telehealth, telemed, teletherapeutic, etc. may be used interchangeably herein.

The term “attribute of a user” may include a quality or feature regarded as a characteristic or inherent part of someone.

The term “attribute of an occupational task” may include that which is regarded as required of and/or desired in any person to be capable of performing an occupational task, such as, without limitation, a physical, cognitive, attentional, or communication capability. One or more attributes may be associated with an occupational task.

Some embodiments are described in connection with thresholds. As used herein, an indication that a threshold condition may be or has been satisfied may refer to a value being greater than a threshold condition, greater than or the same as a threshold condition, the same as a threshold condition, smaller than or the same as a threshold condition, smaller than a threshold condition, above a threshold condition, above or at a threshold condition, at a threshold condition, below or at a threshold condition, below a threshold condition, or the like.

In some embodiments, the threshold condition may be configurable by the patient and/or a healthcare professional.

### DETAILED DESCRIPTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Determining a treatment plan for a patient having certain attributes (e.g., vital-sign or other measurements; performance; demographic; geographic; diagnostic; measurement- or test-based; medically historic; etiologic; cohort-associative; differentially diagnostic; surgical, physically therapeutic, pharmacologic and other treatment(s) recommended; etc.) may be a technically challenging problem. For example, a multitude of information may be considered when determining a treatment plan, which may result in inefficiencies and inaccuracies in the treatment plan selection process. In a rehabilitative setting, some of the multitude of information considered may include attributes of the patient such as personal information, performance information, and measurement information. The personal information may include, e.g., demographic, psychographic or other information, such as an age, a weight, a gender, a height, a body mass index, a medical condition, a familial medication history, an injury, a medical procedure, a medication prescribed, or some combination thereof. The performance information may include, e.g., an elapsed time of using a treatment apparatus, an amount of force exerted on a portion of the treatment apparatus, a range of motion achieved on the treatment apparatus, a movement speed of a portion of the treatment apparatus, an indication of a plurality of pain levels using the treatment apparatus, or some combination thereof. The measurement information may include, e.g., a vital sign, a respiration rate, a heartrate, a temperature, a blood pressure, or some combination thereof. It may be desirable to process the attributes of a multitude of patients, the treatment plans performed for those patients, and the results of the treatment plans for those patients.

Further, another technical problem may involve distally treating, via a computing device during a telemedicine or telehealth session, a patient from a location different from a location at which the patient is located. An additional technical problem is controlling or enabling the control of, from the different location, a treatment apparatus used by the patient at the location at which the patient is located. Oftentimes, when a patient undergoes rehabilitative surgery (e.g., knee surgery), a physical therapist or other healthcare professional may prescribe a treatment apparatus to the patient to use to perform a treatment protocol at their residence or any mobile location or temporary domicile. A healthcare professional may refer to a doctor, physician assistant, nurse, chiropractor, dentist, physical therapist, acupuncturist, physical trainer, or the like. A healthcare professional may refer to any person with a credential, license, degree, or the like in the field of medicine, physical therapy, rehabilitation, or the like.

Since the physical therapist or other healthcare professional is located in a different location from the patient and the treatment apparatus, it may be technically challenging for the physical therapist or other healthcare professional to monitor the patient's actual progress (as opposed to relying on the patient's word about their progress) using the treatment apparatus, modify the treatment plan according to the patient's progress, adapt the treatment apparatus to the personal attributes of the patient as the patient performs the treatment plan, and the like.

Accordingly, embodiments of the present disclosure pertain to using artificial intelligence and/or machine learning to assign patients to cohorts and to dynamically control a treatment apparatus based on the assignment during an adaptive telemedical session. In some embodiments, numerous treatment apparatuses may be provided to patients. The treatment apparatuses may be used by the patients to perform treatment plans in their residences, at a gym, at a rehabilitative center, at a hospital, or any suitable location, including permanent or temporary domiciles. In some embodiments, the treatment apparatuses may be communicatively coupled to a server. Attributes of the patients may be collected before, during, and/or after the patients perform the treatment plans. For example, the personal information, the performance information, and the measurement information may be collected before, during, and/or after the person performs the treatment plans. The results (e.g., improved performance or decreased performance) of performing each exercise may be collected from the treatment apparatus throughout the treatment plan and after the treatment plan is performed. The parameters, settings, configurations, etc. (e.g., position of pedal, amount of resistance, etc.) of the treatment apparatus may be collected before, during, and/or after the treatment plan is performed.

Each attribute of the patient, each result, and each parameter, setting, configuration, etc. may be timestamped and may be correlated with a particular step in the treatment plan. Such a technique may enable determining which steps in the treatment plan lead to desired results (e.g., improved muscle strength, range of motion, etc.) and which steps lead to diminishing returns (e.g., continuing to exercise after 3 minutes actually delays or harms recovery).

Data may be collected from the treatment apparatuses and/or any suitable computing device (e.g., computing devices where personal information is entered, such as a clinician interface or patient interface) over time as the patients use the treatment apparatuses to perform the various treatment plans. The data that may be collected may include the attributes of the patients, the treatment plans performed by the patients, and the results of the treatment plans.

In some embodiments, the data may be processed to group certain people into cohorts. The people may be grouped by people having certain or selected similar attributes, treatment plans, and results of performing the treatment plans. For example, athletic people having no medical conditions who perform a treatment plan (e.g., use the treatment apparatus for 30 minutes a day 5 times a week for 3 weeks) and who fully recover may be grouped into a first cohort. Older people who are classified obese and who perform a treatment plan (e.g., use the treatment plan for 10 minutes a day 3 times a week for 4 weeks) and who improve their range of motion by 75 percent may be grouped into a second cohort.

In some embodiments, an artificial intelligence engine may include one or more machine learning models that are trained using the cohorts. For example, the one or more machine learning models may be trained to receive an input of attributes of a new patient and to output a treatment plan for the patient that results in a desired result. The machine learning models may match a pattern between the attributes of the new patient and at least one patient of the patients included in a particular cohort. When a pattern is matched, the machine learning models may assign the new patient to the particular cohort and select the treatment plan associated with the at least one patient. The artificial intelligence engine may be configured to control, distally and based on the treatment plan, the treatment apparatus while the new patient uses the treatment apparatus to perform the treatment plan.

As may be appreciated, the attributes of the new patient may change as the new patient uses the treatment apparatus to perform the treatment plan. For example, the performance of the patient may improve quicker than expected for people in the cohort to which the new patient is currently assigned. Accordingly, the machine learning models may be trained to dynamically reassign, based on the changed attributes, the new patient to a different cohort that includes people having attributes similar to the now-changed attributes as the new patient. For example, a clinically obese patient may lose weight and no longer meet the weight criterion for the initial cohort, result in the patient being reassigned to a different cohort with a different weight criterion. A different treatment plan may be selected for the new patient, and the treatment apparatus may be controlled, distally and based on the different treatment plan, the treatment apparatus while the new patient uses the treatment apparatus to perform the treatment plan. Such techniques may provide the technical solution of distally controlling a treatment apparatus. Further, the techniques may lead to faster recovery times and/or better results for the patients because the treatment plan that most accurately fits their attributes is selected and implemented, in real-time, at any given moment. “Real-time” may also refer to near real-time, which may be less than 10 seconds. As described herein, the term “results” may refer to medical results or medical outcomes. Results and outcomes may refer to responses to medical actions.

Depending on what result is desired, the artificial intelligence engine may be trained to output several treatment plans. For example, one result may include recovering to a threshold level (e.g., 75% range of motion) in a fastest amount of time, while another result may include fully recovering (e.g., 100% range of motion) regardless of the amount of time. The data obtained from the patients and sorted into cohorts may indicate that a first treatment plan provides the first result for people with attributes similar to the patient's, and that a second treatment plan provides the second result for people with attributes similar to the patient.

Further, the artificial intelligence engine may also be trained to output treatment plans that are not optimal or sub-optimal or even inappropriate (all referred to, without limitation, as “excluded treatment plans”) for the patient. For example, if a patient has high blood pressure, a particular exercise may not be approved or suitable for the patient as it may put the patient at unnecessary risk or even induce a hypertensive crisis and, accordingly, that exercise may be flagged in the excluded treatment plan for the patient.

In some embodiments, the treatment plans and/or excluded treatment plans may be presented, during a telemedicine or telehealth session, to a healthcare professional. The healthcare professional may select a particular treatment plan for the patient to cause that treatment plan to be transmitted to the patient and/or to control, based on the treatment plan, the treatment apparatus. In some embodiments, to facilitate telehealth or telemedicine applications, including remote diagnoses, determination of treatment plans and rehabilitative and/or pharmacologic prescriptions, the artificial intelligence engine may receive and/or operate distally from the patient and the treatment apparatus. In such cases, the recommended treatment plans and/or excluded treatment plans may be presented simultaneously with a video of the patient in real-time or near real-time during a telemedicine or telehealth session on a user interface of a computing device of a healthcare professional. The video may also be accompanied by audio, text and other multimedia information. Real-time may refer to less than or equal to 2 seconds. Near real-time may refer to any interaction of a sufficiently short time to enable two individuals to engage in a dialogue via such user interface, and will generally be less than 10 seconds but greater than 2 seconds.

Presenting the treatment plans generated by the artificial intelligence engine concurrently with a presentation of the patient video may provide an enhanced user interface because the healthcare professional may continue to visually and/or otherwise communicate with the patient while also reviewing the treatment plans on the same user interface. The enhanced user interface may improve the healthcare professional's experience using the computing device and may encourage the healthcare professional to reuse the user interface. Such a technique may also reduce computing resources (e.g., processing, memory, network) because the healthcare professional does not have to switch to another user interface screen to enter a query for a treatment plan to recommend based on the attributes of the patient. The artificial intelligence engine provides, dynamically on the fly, the treatment plans and excluded treatment plans.

In some embodiments, the treatment apparatus may be adaptive and/or personalized because its properties, configurations, and positions may be adapted to the needs of a particular patient. For example, the pedals may be dynamically adjusted on the fly (e.g., via a telemedicine session or based on programmed configurations in response to certain measurements being detected) to increase or decrease a range of motion to comply with a treatment plan designed for the user. In some embodiments, a healthcare professional may adapt, remotely during a telemedicine session, the treatment apparatus to the needs of the patient by causing a control instruction to be transmitted from a server to treatment apparatus. Such adaptive nature may improve the results of recovery for a patient, furthering the goals of personalized medicine, and enabling personalization of the treatment plan on a per-individual basis.

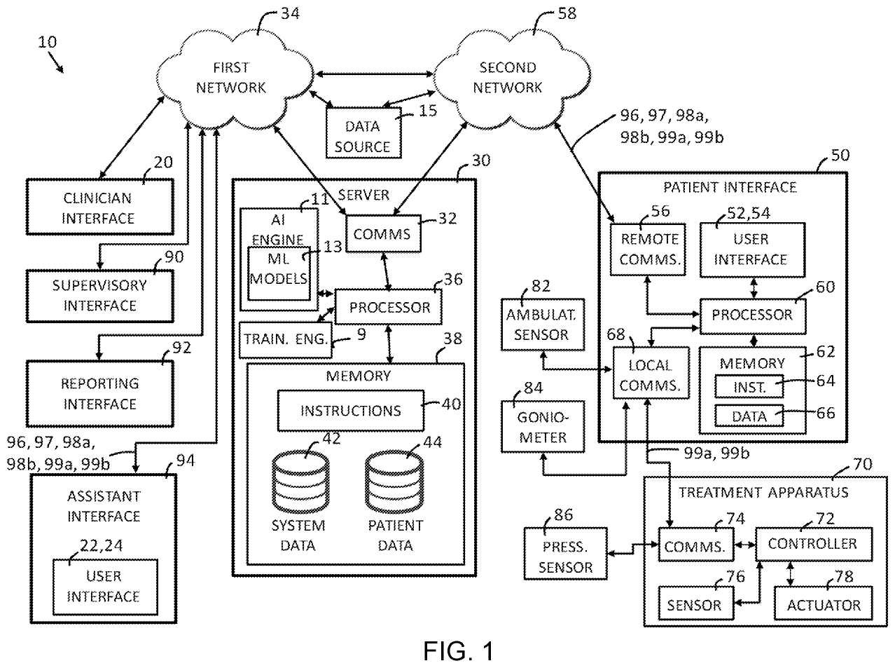

FIG. 1 shows a block diagram of a computer-implemented system 10, hereinafter called “the system” for managing a treatment plan. Managing the treatment plan may include using an artificial intelligence engine to recommend treatment plans and/or provide excluded treatment plans that should not be recommended to a patient.

The system 10 also includes a server 30 configured to store and to provide data related to managing the treatment plan. The server 30 may include one or more computers and may take the form of a distributed and/or virtualized computer or computers. The server 30 also includes a first communication interface 32 configured to communicate with the clinician interface 20 via a first network 34. In some embodiments, the first network 34 may include wired and/or wireless network connections such as Wi-Fi, Bluetooth, ZigBee, Near-Field Communications (NFC), cellular data network, etc. The server 30 includes a first processor 36 and a first machine-readable storage memory 38, which may be called a “memory” for short, holding first instructions 40 for performing the various actions of the server 30 for execution by the first processor 36. The server 30 is configured to store data regarding the treatment plan. For example, the memory 38 includes a system data store 42 configured to hold system data, such as data pertaining to treatment plans for treating one or more patients. The server 30 is also configured to store data regarding performance by a patient in following a treatment plan. For example, the memory 38 includes a patient data store 44 configured to hold patient data, such as data pertaining to the one or more patients, including data representing each patient's performance within the treatment plan.

In addition, the attributes (e.g., personal, performance, measurement, etc.) of the people, the treatment plans followed by the people, the level of compliance with the treatment plans, and the results of the treatment plans may use correlations and other statistical or probabilistic measures to enable the partitioning of or to partition the treatment plans into different patient cohort-equivalent databases in the patient data store 44. For example, the data for a first cohort of first patients having a first similar injury, a first similar medical condition, a first similar medical procedure performed, a first treatment plan followed by the first patient, a first occupational task or tasks, and a first result of the treatment plan may be stored in a first patient database. The data for a second cohort of second patients having a second similar injury, a second similar medical condition, a second similar medical procedure performed, a second treatment plan followed by the second patient, a second occupational task or tasks, and a second result of the treatment plan may be stored in a second patient database. Any single attribute or any combination of attributes may be used to separate the cohorts of patients. In some embodiments, the different cohorts of patients may be stored in different partitions or volumes of the same database. There is no specific limit to the number of different cohorts of patients allowed, other than as limited by mathematical combinatoric and/or partition theory.

This attribute data, occupational task attribute data, treatment plan data, and results data may be obtained from numerous treatment apparatuses and/or computing devices over time and stored in the database 44. The attribute data, occupational task attribute data, treatment plan data, and results data may be correlated in the patient-cohort databases in the patient data store 44. The attributes of the people may include personal information, performance information, and/or measurement information.

In addition to the historical information about other people stored in the patient cohort-equivalent databases, real-time or near-real-time information based on the current patient's attributes about a current patient being treated may be stored in an appropriate patient cohort-equivalent database. The attributes of the patient may be determined to match with or be regarded as similar to the attributes of another person in a particular cohort (e.g., cohort A), and the attributes of occupational tasks associated with the patient may be determined to match with or may be regarded as similar to the attributes of the occupational tasks associated with another person in the particular cohort, and the patient may be assigned to that cohort.

In some embodiments, the server 30 may execute an artificial intelligence (AI) engine 11 that uses one or more machine learning models 13 to perform at least one of the embodiments disclosed herein. The server 30 may include a training engine 9 capable of generating the one or more machine learning models 13. The machine learning models 13 may be trained to assign people to certain cohorts based on their attributes, attributes of occupational tasks associated with the people, select treatment plans using real-time and historical data correlations involving patient cohort-equivalents, and control a treatment apparatus 70, among other things. The one or more machine learning models 13 may be generated by the training engine 9 and may be implemented in computer instructions executable by one or more processing devices of the training engine 9 and/or the servers 30. To generate the one or more machine learning models 13, the training engine 9 may train the one or more machine learning models 13. The one or more machine learning models 13 may be used by the artificial intelligence engine 11.

The training engine 9 may be a rackmount server, a router computer, a personal computer, a portable digital assistant, a smartphone, a laptop computer, a tablet computer, a netbook, a desktop computer, an Internet of Things (IoT) device, any other desired computing device, or any combination of the above. The training engine 9 may be cloud-based or a real-time software platform, and it may include privacy software or protocols, and/or security software or protocols.

To train the one or more machine learning models 13, the training engine 9 may use a training data set of a corpus of the attributes of the people that used the treatment apparatus 70 to perform treatment plans, the attributes of the occupational tasks of the people, the details (e.g., treatment protocol including exercises, amount of time to perform the exercises, how often to perform the exercises, a schedule of exercises, parameters/configurations/settings of the treatment apparatus 70 throughout each step of the treatment plan, etc.) of the treatment plans performed by the people using the treatment apparatus 70, and the results of the treatment plans performed by the people. The one or more machine learning models 13 may be trained to match patterns of attributes of a patient with attributes of other people assigned to a particular cohort and to match patterns of attributes of occupational tasks of the patient with attributes of occupational tasks of other people assigned to the particular cohort.

As used throughout this disclosure, the terms “match”, “match with”, or “matches with” may refer to an exact match, a correlative match, a substantial match, a statistically measured match, etc.

The one or more machine learning models 13 may be trained to receive the attributes of a patient and attributes of occupational tasks associated with the patient as input, map the attributes to attributes of people assigned to a cohort and to attributes of occupational tasks of the people assigned to the cohort, and select a treatment plan from that cohort. The one or more machine learning models 13 may also be trained to control, based on the treatment plan, the machine learning apparatus 70.

Different machine learning models 13 may be trained to recommend different treatment plans for different desired results. For example, one machine learning model may be trained to recommend treatment plans for most effective recovery, while another machine learning model may be trained to recommend treatment plans based on speed of recovery.

Using training data that includes training inputs and corresponding target outputs, the one or more machine learning models 13 may refer to model artifacts created by the training engine 9. The training engine 9 may find patterns in the training data wherein such patterns map the training input to the target output, and generate the machine learning models 13 that capture these patterns. In some embodiments, the artificial intelligence engine 11, the database 33, and/or the training engine 9 may reside on another component (e.g., healthcare professional interface 94, clinician interface 20, etc.) depicted in FIG. 1.

The one or more machine learning models 13 may comprise, e.g., a single level of linear or non-linear operations (e.g., a support vector machine [SVM]) or the machine learning models 13 may be a deep network, i.e., a machine learning model comprising multiple levels of non-linear operations. Examples of deep networks are neural networks including generative adversarial networks, convolutional neural networks, recurrent neural networks with one or more hidden layers, and fully connected neural networks (e.g., each neuron may transmit its output signal to the input of the remaining neurons, as well as to itself). For example, the machine learning model may include numerous layers and/or hidden layers that perform calculations (e.g., dot products) using various neurons.

The system 10 also includes a patient interface 50 configured to communicate information to a patient and to receive feedback from the patient. Specifically, the patient interface includes an input device 52 and an output device 54, which may be collectively called a patient user interface 52, 54. The input device 52 may include one or more devices, such as a keyboard, a mouse, a touch screen input, a gesture sensor, and/or a microphone and processor configured for voice recognition. The output device 54 may take one or more different forms including, for example, a computer monitor or display screen on a tablet, smartphone, or a smart watch. The output device 54 may include other hardware and/or software components such as a projector, virtual reality capability, augmented reality capability, etc. The output device 54 may incorporate various different visual, audio, or other presentation technologies. For example, the output device 54 may include a non-visual display, such as an audio signal, which may include spoken language and/or other sounds such as tones, chimes, and/or melodies, which may signal different conditions and/or directions. The output device 54 may comprise one or more different display screens presenting various data and/or interfaces or controls for use by the patient. The output device 54 may include graphics, which may be presented by a web-based interface and/or by a computer program or application (“app”).

As shown in FIG. 1, the patient interface 50 includes a second communication interface 56, which may also be called a remote communication interface configured to communicate with the server 30 and/or the clinician interface 20 via a second network 58. In some embodiments, the second network 58 may include a local area network (LAN), such as an Ethernet network. In some embodiments, the second network 58 may include the Internet, and communications between the patient interface 50 and the server 30 and/or the clinician interface 20 may be secured via encryption, such as, for example, by using a virtual private network (VPN). In some embodiments, the second network 58 may include wired and/or wireless network connections such as Wi-Fi, Bluetooth, ZigBee, Near-Field Communications (NFC), cellular data network, etc. In some embodiments, the second network 58 may be the same as and/or operationally coupled to the first network 34.

The patient interface 50 includes a second processor 60 and a second machine-readable storage memory 62 holding second instructions 64 for execution by the second processor 60 for performing various actions of patient interface 50. The second machine-readable storage memory 62 also includes a local data store 66 configured to hold data, such as data pertaining to a treatment plan and/or patient data, such as data representing a patient's performance within a treatment plan. The patient interface 50 also includes a local communication interface 68 configured to communicate with various devices for use by the patient in the vicinity of the patient interface 50. The local communication interface 68 may include wired and/or wireless communications. In some embodiments, the local communication interface 68 may include a local wireless network such as Wi-Fi, Bluetooth, ZigBee, Near-Field Communications (NFC), cellular data network, etc.

The system 10 also includes a treatment apparatus 70 configured to be manipulated by the patient and/or to manipulate a body part of the patient for performing activities according to the treatment plan. In some embodiments, the treatment apparatus 70 may take the form of an exercise and rehabilitation apparatus configured to perform and/or to aid in the performance of a rehabilitation regimen, which may be an orthopedic rehabilitation regimen, and the treatment includes rehabilitation of a body part of the patient, such as a joint or a bone or a muscle group. The treatment apparatus 70 may be any suitable medical, rehabilitative, therapeutic, etc. apparatus configured to be controlled distally via another computing device to treat a patient and/or exercise the patient. The treatment apparatus 70 may be an electromechanical machine including one or more weights, an electromechanical bicycle, an electromechanical spin-wheel, a smart-mirror, a treadmill, or the like. The body part may include, for example, a spine, a hand, a foot, a knee, or a shoulder. The body part may include a part of a joint, a bone, or a muscle group, such as one or more vertebrae, a tendon, or a ligament. As shown in FIG. 1, the treatment apparatus 70 includes a controller 72, which may include one or more processors, computer memory, and/or other components. The treatment apparatus 70 also includes a fourth communication interface 74 configured to communicate with the patient interface 50 via the local communication interface 68. The treatment apparatus 70 also includes one or more internal sensors 76 and an actuator 78, such as a motor. The actuator 78 may be used, for example, for moving the patient's body part and/or for resisting forces by the patient.

The internal sensors 76 may measure one or more operating attributes of the treatment apparatus 70 such as, for example, a force a position, a speed, and/or a velocity. In some embodiments, the internal sensors 76 may include a position sensor configured to measure at least one of a linear motion or an angular motion of a body part of the patient. For example, an internal sensor 76 in the form of a position sensor may measure a distance that the patient is able to move a part of the treatment apparatus 70, where such distance may correspond to a range of motion that the patient's body part is able to achieve. In some embodiments, the internal sensors 76 may include a force sensor configured to measure a force applied by the patient. For example, an internal sensor 76 in the form of a force sensor may measure a force or weight the patient is able to apply, using a particular body part, to the treatment apparatus 70.

The system 10 shown in FIG. 1 also includes an ambulation sensor 82, which communicates with the server 30 via the local communication interface 68 of the patient interface 50. The ambulation sensor 82 may track and store a number of steps taken by the patient. In some embodiments, the ambulation sensor 82 may take the form of a wristband, wristwatch, or smart watch. In some embodiments, the ambulation sensor 82 may be integrated within a phone, such as a smartphone.

The system 10 shown in FIG. 1 also includes a goniometer 84, which communicates with the server 30 via the local communication interface 68 of the patient interface 50. The goniometer 84 measures an angle of the patient's body part. For example, the goniometer 84 may measure the angle of flex of a patient's knee or elbow or shoulder.

The system 10 shown in FIG. 1 also includes a pressure sensor 86, which communicates with the server 30 via the local communication interface 68 of the patient interface 50. The pressure sensor 86 measures an amount of pressure or weight applied by a body part of the patient. For example, pressure sensor 86 may measure an amount of force applied by a patient's foot when pedaling a stationary bike.

The system 10 shown in FIG. 1 also includes a supervisory interface 90 which may be similar or identical to the clinician interface 20. In some embodiments, the supervisory interface 90 may have enhanced functionality beyond what is provided on the clinician interface 20. The supervisory interface 90 may be configured for use by a person having responsibility for the treatment plan, such as an orthopedic surgeon.

The system 10 shown in FIG. 1 also includes a reporting interface 92 which may be similar or identical to the clinician interface 20. In some embodiments, the reporting interface 92 may have less functionality from what is provided on the clinician interface 20. For example, the reporting interface 92 may not have the ability to modify a treatment plan. Such a reporting interface 92 may be used, for example, by a biller to determine the use of the system 10 for billing purposes. In another example, the reporting interface 92 may not have the ability to display patient identifiable information, presenting only pseudonymized data and/or anonymized data for certain data fields concerning a data subject and/or for certain data fields concerning a quasi-identifier of the data subject. Such a reporting interface 92 may be used, for example, by a researcher to determine various effects of a treatment plan on different patients.

The system 10 includes a healthcare professional interface 94 for a healthcare professional, such as a doctor, a nurse, a physical therapist, or a technician, to remotely communicate with the patient interface 50 and/or the treatment apparatus 70. Such remote communications may enable the healthcare professional to provide assistance or guidance to a patient using the system 10. More specifically, the healthcare professional interface 94 is configured to communicate a telemedicine signal 96, 97, 98a, 98b, 99a, 99b with the patient interface 50 via a network connection such as, for example, via the first network 34 and/or the second network 58. The telemedicine signal 96, 97, 98a, 98b, 99a, 99b comprises one of an audio signal 96, an audiovisual signal 97, an interface control signal 98a for controlling a function of the patient interface 50, an interface monitor signal 98b for monitoring a status of the patient interface 50, an apparatus control signal 99a for changing an operating parameter of the treatment apparatus 70, and/or an apparatus monitor signal 99b for monitoring a status of the treatment apparatus 70. In some embodiments, each of the control signals 98a, 99a may be unidirectional, conveying commands from the healthcare professional interface 94 to the patient interface 50. In some embodiments, in response to successfully receiving a control signal 98a, 99a and/or to communicate successful and/or unsuccessful implementation of the requested control action, an acknowledgement message may be sent from the patient interface 50 to the healthcare professional interface 94. In some embodiments, each of the monitor signals 98b, 99b may be unidirectional, status-information commands from the patient interface 50 to the healthcare professional interface 94. In some embodiments, an acknowledgement message may be sent from the healthcare professional interface 94 to the patient interface 50 in response to successfully receiving one of the monitor signals 98b, 99b.

In some embodiments, the patient interface 50 may be configured as a pass-through for the apparatus control signals 99a and the apparatus monitor signals 99b between the treatment apparatus 70 and one or more other devices, such as the healthcare professional interface 94 and/or the server 30. For example, the patient interface 50 may be configured to transmit an apparatus control signal 99a in response to an apparatus control signal 99a within the telemedicine signal 96, 97, 98a, 98b, 99a, 99b from the healthcare professional interface 94.

In some embodiments, the healthcare professional interface 94 may be presented on a shared physical device as the clinician interface 20. For example, the clinician interface 20 may include one or more screens that implement the healthcare professional interface 94. Alternatively or additionally, the clinician interface 20 may include additional hardware components, such as a video camera, a speaker, and/or a microphone, to implement aspects of the healthcare professional interface 94.

In some embodiments, one or more portions of the telemedicine signal 96, 97, 98a, 98b, 99a, 99b may be generated from a prerecorded source (e.g., an audio recording, a video recording, or an animation) for presentation by the output device 54 of the patient interface 50. For example, a tutorial video may be streamed from the server 30 and presented upon the patient interface 50. Content from the prerecorded source may be requested by the patient via the patient interface 50. Alternatively, via a control on the healthcare professional interface 94, the healthcare professional may cause content from the prerecorded source to be played on the patient interface 50.

The healthcare professional interface 94 includes a healthcare professional input device 22 and a healthcare professional display 24, which may be collectively called a healthcare professional user interface 22, 24. The healthcare professional input device 22 may include one or more of a telephone, a keyboard, a mouse, a trackpad, or a touch screen, for example. Alternatively or additionally, the healthcare professional input device 22 may include one or more microphones. In some embodiments, the one or more microphones may take the form of a telephone handset, headset, or wide-area microphone or microphones configured for the healthcare professional to speak to a patient via the patient interface 50. In some embodiments, healthcare professional input device 22 may be configured to provide voice-based functionalities, with hardware and/or software configured to interpret spoken instructions by the healthcare professional by using the one or more microphones. The healthcare professional input device 22 may include functionality provided by or similar to existing voice-based assistants such as Siri by Apple, Alexa by Amazon, Google Assistant, or Bixby by Samsung. The healthcare professional input device 22 may include other hardware and/or software components. The healthcare professional input device 22 may include one or more general-purpose devices and/or special-purpose devices.

The healthcare professional display 24 may take one or more different forms including, for example, a computer monitor or display screen on a tablet, a smartphone, or a smart watch. The healthcare professional display 24 may include other hardware and/or software components such as projectors, virtual reality capabilities, or augmented reality capabilities, etc. The healthcare professional display 24 may incorporate various different visual, audio, or other presentation technologies. For example, the healthcare professional display 24 may include a non-visual display, such as an audio signal, which may include spoken language and/or other sounds such as tones, chimes, melodies, and/or compositions, which may signal different conditions and/or directions. The healthcare professional display 24 may comprise one or more different display screens presenting various data and/or interfaces or controls for use by the healthcare professional. The healthcare professional display 24 may include graphics, which may be presented by a web-based interface and/or by a computer program or application (App.).

In some embodiments, the system 10 may provide computer translation of language from the healthcare professional interface 94 to the patient interface 50 and/or vice-versa. The computer translation of language may include computer translation of spoken language and/or computer translation of text. Additionally or alternatively, the system 10 may provide voice recognition and/or spoken pronunciation of text. For example, the system 10 may convert spoken words to printed text and/or the system 10 may audibly speak language from printed text. The system 10 may be configured to recognize spoken words by any or all of the patient, the clinician, and/or the healthcare professional. In some embodiments, the system 10 may be configured to recognize and react to spoken requests or commands by the patient. For example, the system 10 may automatically initiate a telemedicine session in response to a verbal command by the patient (which may be given in any one of several different languages).

In some embodiments, the server 30 may generate aspects of the healthcare professional display 24 for presentation by the healthcare professional interface 94. For example, the server 30 may include a web server configured to generate the display screens for presentation upon the healthcare professional display 24. For example, the artificial intelligence engine 11 may generate recommended treatment plans and/or excluded treatment plans for patients and generate the display screens including those recommended treatment plans and/or external treatment plans for presentation on the healthcare professional display 24 of the healthcare professional interface 94. In some embodiments, the healthcare professional display 24 may be configured to present a virtualized desktop hosted by the server 30. In some embodiments, the server 30 may be configured to communicate with the healthcare professional interface 94 via the first network 34. In some embodiments, the first network 34 may include a local area network (LAN), such as an Ethernet network. In some embodiments, the first network 34 may include the Internet, and communications between the server 30 and the healthcare professional interface 94 may be secured via privacy enhancing technologies, such as, for example, by using encryption over a virtual private network (VPN). Alternatively or additionally, the server 30 may be configured to communicate with the healthcare professional interface 94 via one or more networks independent of the first network 34 and/or other communication means, such as a direct wired or wireless communication channel. In some embodiments, the patient interface 50 and the treatment apparatus 70 may each operate from a patient location geographically separate from a location of the healthcare professional interface 94. For example, the patient interface 50 and the treatment apparatus 70 may be used as part of an in-home rehabilitation system, which may be aided remotely by using the healthcare professional interface 94 at a centralized location, such as a clinic or a call center.

In some embodiments, the healthcare professional interface 94 may be one of several different terminals (e.g., computing devices) that may be grouped together, for example, in one or more call centers or at one or more clinicians' offices. In some embodiments, a plurality of healthcare professional interfaces 94 may be distributed geographically. In some embodiments, a person may work as a healthcare professional remotely from any conventional office infrastructure. Such remote work may be performed, for example, where the healthcare professional interface 94 takes the form of a computer and/or telephone. This remote work functionality may allow for work-from-home arrangements that may include part time and/or flexible work hours for a healthcare professional.

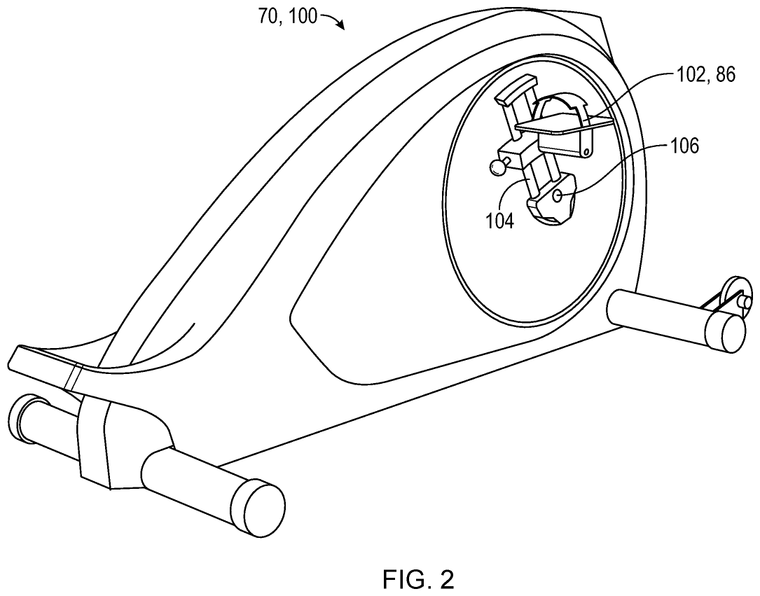

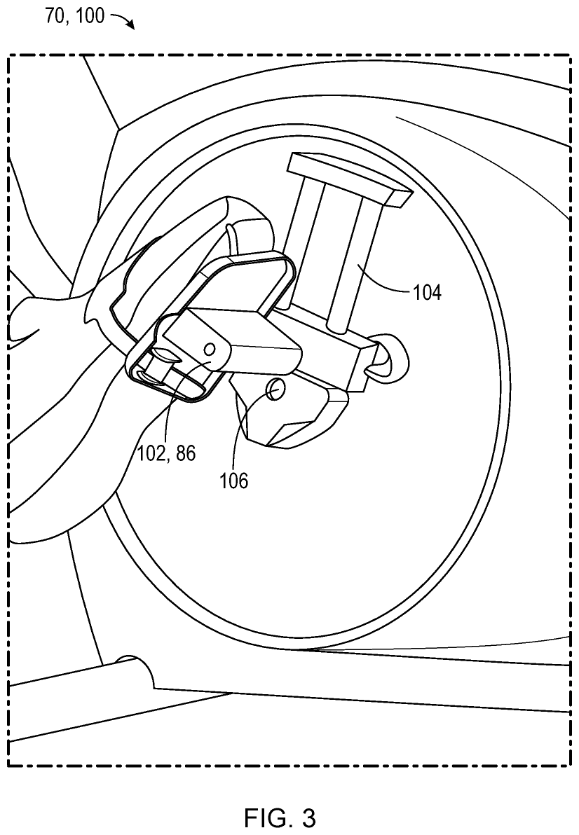

FIGS. 2-3 show an embodiment of a treatment apparatus 70. More specifically, FIG. 2 shows a treatment apparatus 70 in the form of a stationary cycling machine 100, which may be called a stationary bike, for short. The stationary cycling machine 100 includes a set of pedals 102 each attached to a pedal arm 104 for rotation about an axle 106. In some embodiments, and as shown in FIG. 2, the pedals 102 are movable on the pedal arms 104 in order to adjust a range of motion used by the patient in pedaling. For example, the pedals being located inwardly toward the axle 106 corresponds to a smaller range of motion than when the pedals are located outwardly away from the axle 106. A pressure sensor 86 is attached to or embedded within one of the pedals 102 for measuring an amount of force applied by the patient on the pedal 102. The pressure sensor 86 may communicate wirelessly to the treatment apparatus 70 and/or to the patient interface 50.

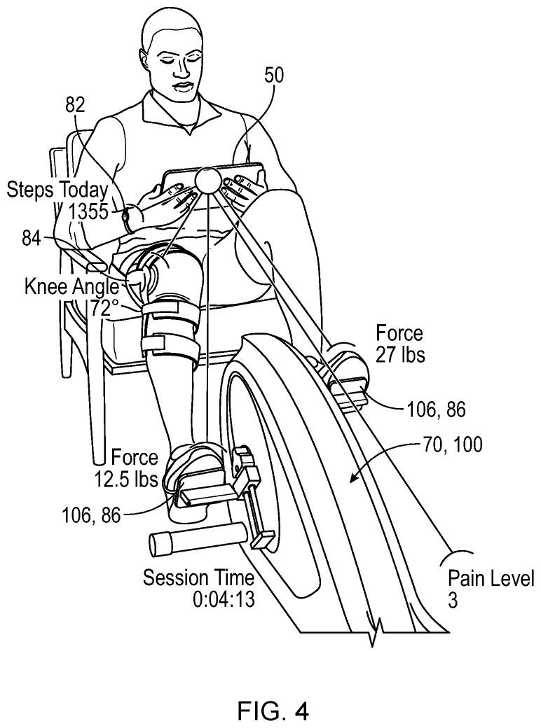

FIG. 4 shows a person (a patient) using the treatment apparatus of FIG. 2, and showing sensors and various data parameters connected to a patient interface 50. The example patient interface 50 is a tablet computer or smartphone, or a phablet, such as an iPad, an iPhone, an Android device, or a Surface tablet, which is held manually by the patient. In some other embodiments, the patient interface 50 may be embedded within or attached to the treatment apparatus 70. FIG. 4 shows the patient wearing the ambulation sensor 82 on his wrist, with a note showing “STEPS TODAY 1355”, indicating that the ambulation sensor 82 has recorded and transmitted that step count to the patient interface 50. FIG. 4 also shows the patient wearing the goniometer 84 on his right knee, with a note showing “KNEE ANGLE 72°”, indicating that the goniometer 84 is measuring and transmitting that knee angle to the patient interface 50. FIG. 4 also shows a right side of one of the pedals 102 with a pressure sensor 86 showing “FORCE 12.5 lbs.,” indicating that the right pedal pressure sensor 86 is measuring and transmitting that force measurement to the patient interface 50. FIG. 4 also shows a left side of one of the pedals 102 with a pressure sensor 86 showing “FORCE 27 lbs.”, indicating that the left pedal pressure sensor 86 is measuring and transmitting that force measurement to the patient interface 50. FIG. 4 also shows other patient data, such as an indicator of “SESSION TIME 0:04:13”, indicating that the patient has been using the treatment apparatus 70 for 4 minutes and 13 seconds. This session time may be determined by the patient interface 50 based on information received from the treatment apparatus 70. FIG. 4 also shows an indicator showing “PAIN LEVEL 3”. Such a pain level may be obtained from the patent in response to a solicitation, such as a question, presented upon the patient interface 50.

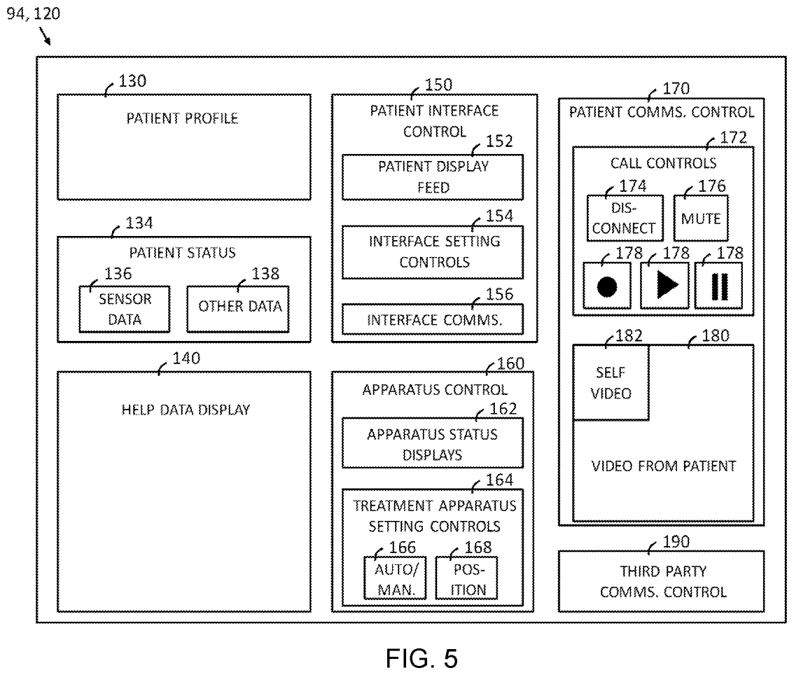

FIG. 5 is an example embodiment of an overview display 120 of the healthcare professional interface 94. Specifically, the overview display 120 presents several different controls and interfaces for the healthcare professional to remotely assist a patient with using the patient interface 50 and/or the treatment apparatus 70. This remote assistance functionality may also be called telemedicine or telehealth.

Specifically, the overview display 120 includes a patient profile display 130 presenting biographical information regarding a patient using the treatment apparatus 70. The patient profile display 130 may take the form of a portion or region of the overview display 120, as shown in FIG. 5, although the patient profile display 130 may take other forms, such as a separate screen or a popup window. In some embodiments, the patient profile display 130 may include a limited subset of the patient's biographical information. More specifically, the data presented upon the patient profile display 130 may depend upon the healthcare professional's need for that information. For example, a healthcare professional that is assisting the patient with a medical issue may be provided with medical history information regarding the patient, whereas a technician troubleshooting an issue with the treatment apparatus 70 may be provided with a much more limited set of information regarding the patient. The technician, for example, may be given only the patient's name. The patient profile display 130 may include pseudonymized data and/or anonymized data or use any privacy enhancing technology to prevent confidential patient data from being communicated in a way that could violate patient confidentiality requirements. Such privacy enhancing technologies may enable compliance with laws, regulations, or other rules of governance such as, but not limited to, the Health Insurance Portability and Accountability Act (HIPAA), or the General Data Protection Regulation (GDPR), wherein the patient may be deemed a “data subject”.

In some embodiments, the patient profile display 130 may present information regarding the treatment plan for the patient to follow in using the treatment apparatus 70. Such treatment plan information may be limited to a healthcare professional who has a certain level of authority, such as a doctor or physical therapist. For example, a healthcare professional who is a doctor assisting the patient with an issue regarding the treatment regimen may be provided with treatment plan information, whereas a technician troubleshooting an issue with the treatment apparatus 70 may not be provided with any information regarding the patient's treatment plan.

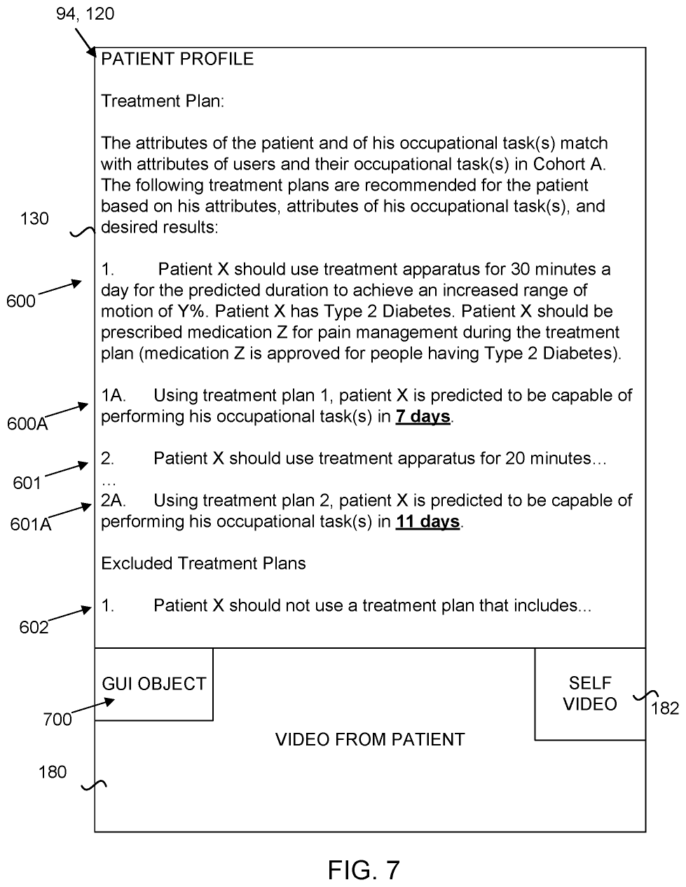

In some embodiments, one or more recommended treatment plans with corresponding predicted estimates of when the patient would be capable of performing the occupational task(s) associated with the patient and/or excluded treatment plans may be presented in the patient profile display 130 to the healthcare professional. The one or more recommended treatment plans with predicted estimates and/or excluded treatment plans may be generated by the artificial intelligence engine 11 of the server 30 and received from the server 30 in real-time during, inter alia, a telemedicine or telehealth session. An example of presenting the one or more recommended treatment plans and predicted estimates and/or excluded treatment plans is described below with reference to FIG. 7.

The example overview display 120 shown in FIG. 5 also includes a patient status display 134 presenting status information regarding a patient using the treatment apparatus 70. The patient status display 134 may take the form of a portion or region of the overview display 120, as shown in FIG. 5, although the patient status display 134 may take other forms, such as a separate screen or a popup window. The patient status display 134 includes sensor data 136 from one or more of the external sensors 82, 84, 86, and/or from one or more internal sensors 76 of the treatment apparatus 70. In some embodiments, the patient status display 134 may present other data 138 regarding the patient, such as last reported pain level, or progress within a treatment plan.

User access controls may be used to limit access, including what data is available to be viewed and/or modified, on any or all of the user interfaces 20, 50, 90, 92, 94 of the system 10. In some embodiments, user access controls may be employed to control what information is available to any given person using the system 10. For example, data presented on the healthcare professional interface 94 may be controlled by user access controls, with permissions set depending on the healthcare professional/user's need for and/or qualifications to view that information.

The example overview display 120 shown in FIG. 5 also includes a help data display 140 presenting information for the healthcare professional to use in assisting the patient. The help data display 140 may take the form of a portion or region of the overview display 120, as shown in FIG. 5. The help data display 140 may take other forms, such as a separate screen or a popup window. The help data display 140 may include, for example, presenting answers to frequently asked questions regarding use of the patient interface 50 and/or the treatment apparatus 70. The help data display 140 may also include research data or best practices. In some embodiments, the help data display 140 may present scripts for answers or explanations in response to patient questions. In some embodiments, the help data display 140 may present flow charts or walk-throughs for the healthcare professional to use in determining a root cause and/or solution to a patient's problem. In some embodiments, the healthcare professional interface 94 may present two or more help data displays 140, which may be the same or different, for simultaneous presentation of help data for use by the healthcare professional. for example, a first help data display may be used to present a troubleshooting flowchart to determine the source of a patient's problem, and a second help data display may present script information for the healthcare professional to read to the patient, such information to preferably include directions for the patient to perform some action, which may help to narrow down or solve the problem. In some embodiments, based upon inputs to the troubleshooting flowchart in the first help data display, the second help data display may automatically populate with script information.

The example overview display 120 shown in FIG. 5 also includes a patient interface control 150 presenting information regarding the patient interface 50, and/or to modify one or more settings of the patient interface 50. The patient interface control 150 may take the form of a portion or region of the overview display 120, as shown in FIG. 5. The patient interface control 150 may take other forms, such as a separate screen or a popup window. The patient interface control 150 may present information communicated to the healthcare professional interface 94 via one or more of the interface monitor signals 98b. As shown in FIG. 5, the patient interface control 150 includes a display feed 152 of the display presented by the patient interface 50. In some embodiments, the display feed 152 may include a live copy of the display screen currently being presented to the patient by the patient interface 50. In other words, the display feed 152 may present an image of what is presented on a display screen of the patient interface 50. In some embodiments, the display feed 152 may include abbreviated information regarding the display screen currently being presented by the patient interface 50, such as a screen name or a screen number. The patient interface control 150 may include a patient interface setting control 154 for the healthcare professional to adjust or to control one or more settings or aspects of the patient interface 50. In some embodiments, the patient interface setting control 154 may cause the healthcare professional interface 94 to generate and/or to transmit an interface control signal 98 for controlling a function or a setting of the patient interface 50.

In some embodiments, the patient interface setting control 154 may include collaborative browsing or co-browsing capability for the healthcare professional to remotely view and/or control the patient interface 50. For example, the patient interface setting control 154 may enable the healthcare professional to remotely enter text to one or more text entry fields on the patient interface 50 and/or to remotely control a cursor on the patient interface 50 using a mouse or touchscreen of the healthcare professional interface 94.

In some embodiments, using the patient interface 50, the patient interface setting control 154 may allow the healthcare professional to change a setting that cannot be changed by the patient. For example, the patient interface 50 may be precluded from accessing a language setting to prevent a patient from inadvertently switching, on the patient interface 50, the language used for the displays, whereas the patient interface setting control 154 may enable the healthcare professional to change the language setting of the patient interface 50. In another example, the patient interface 50 may not be able to change a font size setting to a smaller size in order to prevent a patient from inadvertently switching the font size used for the displays on the patient interface 50 such that the display would become illegible to the patient, whereas the patient interface setting control 154 may provide for the healthcare professional to change the font size setting of the patient interface 50.

The example overview display 120 shown in FIG. 5 also includes an interface communications display 156 showing the status of communications between the patient interface 50 and one or more other devices 70, 82, 84, such as the treatment apparatus 70, the ambulation sensor 82, and/or the goniometer 84. The interface communications display 156 may take the form of a portion or region of the overview display 120, as shown in FIG. 5. The interface communications display 156 may take other forms, such as a separate screen or a popup window. The interface communications display 156 may include controls for the healthcare professional to remotely modify communications with one or more of the other devices 70, 82, 84. For example, the healthcare professional may remotely command the patient interface 50 to reset communications with one of the other devices 70, 82, 84, or to establish communications with a new one of the other devices 70, 82, 84. This functionality may be used, for example, where the patient has a problem with one of the other devices 70, 82, 84, or where the patient receives a new or a replacement one of the other devices 70, 82, 84.

The example overview display 120 shown in FIG. 5 also includes an apparatus control 160 for the healthcare professional to view and/or to control information regarding the treatment apparatus 70. The apparatus control 160 may take the form of a portion or region of the overview display 120, as shown in FIG. 5. The apparatus control 160 may take other forms, such as a separate screen or a popup window. The apparatus control 160 may include an apparatus status display 162 with information regarding the current status of the apparatus. The apparatus status display 162 may present information communicated to the healthcare professional interface 94 via one or more of the apparatus monitor signals 99b. The apparatus status display 162 may indicate whether the treatment apparatus 70 is currently communicating with the patient interface 50. The apparatus status display 162 may present other current and/or historical information regarding the status of the treatment apparatus 70.

The apparatus control 160 may include an apparatus setting control 164 for the healthcare professional to adjust or control one or more aspects of the treatment apparatus 70. The apparatus setting control 164 may cause the healthcare professional interface 94 to generate and/or to transmit an apparatus control signal 99 for changing an operating parameter of the treatment apparatus 70, (e.g., a pedal radius setting, a resistance setting, a target RPM, etc.). The apparatus setting control 164 may include a mode button 166 and a position control 168, which may be used in conjunction for the healthcare professional to place an actuator 78 of the treatment apparatus 70 in a manual mode, after which a setting, such as a position or a speed of the actuator 78, can be changed using the position control 168. The mode button 166 may provide for a setting, such as a position, to be toggled between automatic and manual modes. In some embodiments, one or more settings may be adjustable at any time, and without having an associated auto/manual mode. In some embodiments, the healthcare professional may change an operating parameter of the treatment apparatus 70, such as a pedal radius setting, while the patient is actively using the treatment apparatus 70. Such “on the fly” adjustment may or may not be available to the patient using the patient interface 50. In some embodiments, the apparatus setting control 164 may allow the healthcare professional to change a setting that cannot be changed by the patient using the patient interface 50. For example, the patient interface 50 may be precluded from changing a preconfigured setting, such as a height or a tilt setting of the treatment apparatus 70, whereas the apparatus setting control 164 may provide for the healthcare professional to change the height or tilt setting of the treatment apparatus 70.

The example overview display 120 shown in FIG. 5 also includes a patient communications control 170 for controlling an audio or an audiovisual communications session with the patient interface 50. The communications session with the patient interface 50 may comprise a live feed from the healthcare professional interface 94 for presentation by the output device of the patient interface 50. The live feed may take the form of an audio feed and/or a video feed. In some embodiments, the patient interface 50 may be configured to provide two-way audio or audiovisual communications with a person using the healthcare professional interface 94. Specifically, the communications session with the patient interface 50 may include bidirectional (two-way) video or audiovisual feeds, with each of the patient interface 50 and the healthcare professional interface 94 presenting video of the other one. In some embodiments, the patient interface 50 may present video from the healthcare professional interface 94, while the healthcare professional interface 94 presents only audio or the healthcare professional interface 94 presents no live audio or visual signal from the patient interface 50. In some embodiments, the healthcare professional interface 94 may present video from the patient interface 50, while the patient interface 50 presents only audio or the patient interface 50 presents no live audio or visual signal from the healthcare professional interface 94.

In some embodiments, the audio or an audiovisual communications session with the patient interface 50 may take place, at least in part, while the patient is performing the rehabilitation regimen upon the body part. The patient communications control 170 may take the form of a portion or region of the overview display 120, as shown in FIG. 5. The patient communications control 170 may take other forms, such as a separate screen or a popup window. The audio and/or audiovisual communications may be processed and/or directed by the healthcare professional interface 94 and/or by another device or devices, such as a telephone system, or a videoconferencing system used by the healthcare professional while the healthcare professional uses the healthcare professional interface 94. Alternatively or additionally, the audio and/or audiovisual communications may include communications with a third party. For example, the system 10 may enable the healthcare professional to initiate a 3-way conversation regarding use of a particular piece of hardware or software, with the patient and a subject matter expert, such as a healthcare professional or a specialist. The example patient communications control 170 shown in FIG. 5 includes call controls 172 for the healthcare professional to use in managing various aspects of the audio or audiovisual communications with the patient. The call controls 172 include a disconnect button 174 for the healthcare professional to end the audio or audiovisual communications session. The call controls 172 also include a mute button 176 to temporarily silence an audio or audiovisual signal from the healthcare professional interface 94. In some embodiments, the call controls 172 may include other features, such as a hold button (not shown). The call controls 172 also include one or more record/playback controls 178, such as record, play, and pause buttons to control, with the patient interface 50, recording and/or playback of audio and/or video from the teleconference session. The call controls 172 also include a video feed display 180 for presenting still and/or video images from the patient interface 50, and a self-video display 182 showing the current image of the healthcare professional using the healthcare professional interface. The self-video display 182 may be presented as a picture-in-picture format, within a section of the video feed display 180, as shown in FIG. 5. Alternatively or additionally, the self-video display 182 may be presented separately and/or independently from the video feed display 180.

The example overview display 120 shown in FIG. 5 also includes a third party communications control 190 for use in conducting audio and/or audiovisual communications with a third party. The third party communications control 190 may take the form of a portion or region of the overview display 120, as shown in FIG. 5. The third party communications control 190 may take other forms, such as a display on a separate screen or a popup window. The third party communications control 190 may include one or more controls, such as a contact list and/or buttons or controls to contact a third party regarding use of a particular piece of hardware or software, e.g., a subject matter expert, such as a healthcare professional or a specialist. The third party communications control 190 may include conference calling capability for the third party to simultaneously communicate with both the healthcare professional via the healthcare professional interface 94, and with the patient via the patient interface 50. For example, the system 10 may provide for the healthcare professional to initiate a 3-way conversation with the patient and the third party.

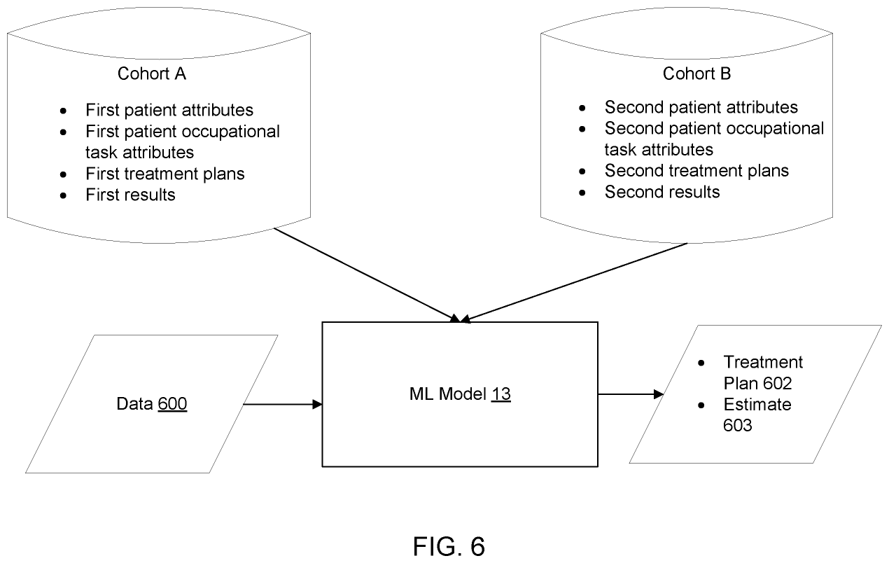

FIG. 6 shows an example block diagram of training a machine learning model 13 to output, based on data 600 pertaining to the patient and to the occupational task(s) associated with the patient, a treatment plan 602 for the patient and a predicted estimate 603 of when the patient performing treatment plan 602 would be capable of performing the occupational task(s) associated with the patient, according to the present disclosure. Data pertaining to other patients may be received by the server 30. The other patients may have used various treatment apparatuses to perform treatment plans. The data may include attributes of the other patients, attributes of occupational tasks of the other patients, the details of the treatment plans performed by the other patients, and/or the results of performing the treatment plans (e.g., a percent of recovery of a portion of the patients' bodies, an amount of recovery of a portion of the patients' bodies, an amount of increase or decrease in muscle strength of a portion of patients' bodies, an amount of increase or decrease in range of motion of a portion of patients' bodies, etc.).

As depicted, the data has been assigned to different cohorts. Cohort A includes data for patients having similar first attributes, first attributes of occupational tasks, first treatment plans, and first results. Cohort B includes data for patients having similar second attributes, second attributes of occupational tasks, second treatment plans, and second results. For example, cohort A may include first attributes of patients in their twenties without any medical conditions who tore a leg ligament while lifting boxes from trucks in a warehouse; their treatment plans may include a certain treatment protocol (e.g., use the treatment apparatus 70 for 30 minutes 5 times a week for a predicted estimate of time, wherein values for the properties, configurations, and/or settings of the treatment apparatus 70 are set to X (where X is a numerical value) for a first portion of the predicted estimate of time and to Y (where Y is a numerical value) for a second portion of the predicted estimate of time).

Cohort A and cohort B may be included in a training dataset used to train the machine learning model 13. The machine learning model 13 may be trained to match a pattern between attributes for each cohort and output the treatment plan that provides the result, and the corresponding predicted estimate. Accordingly, when the data 600 for a new patient is input into the trained machine learning model 13, the trained machine learning model 13 may match the attributes included in the data 600 with attributes in either cohort A or cohort B and output the appropriate treatment plan 602 and the corresponding predicted estimate 603. In some embodiments, the machine learning model 13 may be trained to output one or more excluded treatment plans that should not be performed by the new patient.

FIG. 7 shows an embodiment of an overview display 120 of the healthcare professional interface 94 presenting recommended treatment plans and any excluded treatment plans in real-time during a telemedicine session, as well as predictions of estimates of when a patient, upon following a respective treatment plan, would be capable of performing the patient's occupational tasks, according to the present disclosure. As depicted, the overview display 120 just includes sections for the patient profile 130 and the video feed display 180, including the self-video display 182. Any suitable configuration of controls and interfaces of the overview display 120 described with reference to FIG. 5 may be presented in addition to or instead of the patient profile 130, the video feed display 180, and the self-video display 182.

The healthcare professional using the healthcare professional interface 94 (e.g., computing device) during the telemedicine session may be presented in the self-video 182 in a portion of the overview display 120 (e.g., user interface presented on a display screen 24 of the healthcare professional interface 94) that also presents a video from the patient in the video feed display 180. Further, the video feed display 180 may also include a graphical user interface (GUI) object 700 (e.g., a button) that enables the healthcare professional to share, in real-time or near real-time during the telemedicine session, the recommended treatment plans and/or the predicted estimates and/or the excluded treatment plans with the patient on the patient interface 50. The healthcare professional may select the GUI object 700 to share the recommended treatment plans and/or the excluded treatment plans. The healthcare professional may select the GUI object 700 to share the predicted estimates for the recommended treatment plans. As depicted, another portion of the overview display 120 includes the patient profile display 130.

The patient profile display 130 presents two example recommended treatment plans 600, 601 (in part) each with example predicted estimates 600A, 601A of when the patient performing a respective recommended treatment plan 600, 601 would be capable of performing his occupational tasks. Also, the patient profile display 130 presents one example excluded treatment plan 602 (in part). As described herein, the treatment plans may be recommended in view of attributes of the patient being treated and in view of at least one attribute of an occupational task associated with the patient. The occupational task may be one of multiple occupational tasks for an occupation of the patient. To generate the recommended treatment plans 600, 601 the patient should follow in order for the patient to achieve a desired result, a pattern between the attributes of the patient being treated, the attributes of the occupational tasks associated with the patient, and a cohort of other people and their occupational tasks who have used the treatment apparatus 70 to perform a treatment plan may be matched by one or more machine learning models 13 of the artificial intelligence engine 11. Each of the recommended treatment plans may be generated based on different desired results. A desired result may be to first achieve a patient's capability of performing one particular occupational task of a set of occupational tasks. For example, a desired result may be for the patient to first achieve a flexibility requirement, and for the patient to later achieve a strength requirement. A different desired result may be to improve a patient's capabilities to perform some or all occupational tasks at about the same rate. For example, a desired result may be for the patient to achieve both a flexibility requirement and a strength requirement at about the same time.

For example, as depicted, the patient profile display 130 presents “The attributes of the patient and of his occupational task(s) match with attributes of users and their occupational task(s) in Cohort A. The following treatment plans are recommended for the patient based on his attributes and attributes of his occupational task(s).” Then, the patient profile display 130 presents recommended treatment plans from cohort A, and each treatment plan provides different results.

As depicted, treatment plan 600 indicates “Patient X should use treatment apparatus for 30 minutes a day for the predicted duration to achieve an increased range of motion of Y %; Patient X has Type 2 Diabetes. Patient X should be prescribed medication Z for pain management during the treatment plan (medication Z is approved for people having Type 2 Diabetes).” Accordingly, treatment plan 600 is for achieving an increase in the range of motion of Y %. As may be appreciated, treatment plan 600 also includes a recommended medication (e.g., medication Z) to prescribe to the patient to manage pain in view of a known medical disease (e.g., Type 2 Diabetes) of the patient. That is, the recommended patient medication not only does not conflict with the medical condition of the patient but thereby improves the probability of a superior patient outcome. This specific example and all such examples elsewhere herein are not intended to limit in any way the generated treatment plan from recommending multiple medications, or from handling the acknowledgement, view, diagnosis and/or treatment of comorbid conditions or diseases.

As depicted, a predicted estimate 600A corresponding to treatment plan 600 indicates a time at which the patient performing treatment plan 600 would be capable of performing the occupational task(s) associated with the patient: “Using treatment plan 1, patient X is predicted to be capable of performing his occupational task(s) in 7 days.”

Recommended treatment plan 601 may specify, based on a different desired result of the treatment plan, a different treatment plan, including a different treatment protocol for a treatment apparatus, a different medication regimen, etc. Furthermore, predicted estimate 601A corresponding to treatment plan 601 may indicate a respective time at which the patient performing treatment plan 601 would be capable of performing the occupational task(s) associated with the patient.

It will be appreciated that an estimate of when a patient performing a respective treatment plan would be capable of performing a particular occupational task or particular occupational tasks may be depicted in the form of time units such as days, weeks and/or months, half-days, half-weeks, half-months, or combinations thereof; or of percentages or portions of any of the foregoing, and any references herein to “time units” shall be deemed to include embodiments wherein the time unit is a percentage or portion. An estimate may alternatively be depicted in the form of a particular date, such as “Jul. 31, 2022”, calculated on the basis of a starting date and a particular time unit predicted via the artificial intelligence engine. Alternative forms of depiction of a predicted estimate may be available. Furthermore, a user may be provided with the option to toggle between different alternative forms of depiction of a predicted estimate, such as via selection through GUI OBJECT 700.

As depicted, the patient profile display 130 may also present the excluded treatment plans 602. These types of treatment plans are shown to the healthcare professional using the healthcare professional interface 94 to alert the healthcare professional not to recommend certain portions of a treatment plan to the patient. For example, an excluded treatment plan 602 could specify the following: “Patient X should not use treatment apparatus for longer than 30 minutes a day due to a heart condition; Patient X has Type 2 Diabetes; and Patient X should not be prescribed medication M for pain management during the treatment plan (in this scenario, medication M can cause complications for people having Type 2 Diabetes). Specifically, the excluded treatment plan 602 points out a limitation of a treatment protocol where, due to a heart condition, Patient X should not exercise for more than 30 minutes a day. The ruled-out treatment plan also points out that Patient X should not be prescribed medication M because it conflicts with the medical condition Type 2 Diabetes.

The healthcare professional may select the treatment plan for the patient on the overview display 120. For example, the healthcare professional may use an input peripheral (e.g., mouse, touchscreen, microphone, keyboard, etc.) to select from the treatment plans 600, 601 for the patient. In some embodiments, during the telemedicine session, the healthcare professional may discuss the pros and cons of the recommended treatment plans 600, 601 with the patient. In some embodiments, during the telemedicine session, the healthcare professional may discuss the predicted estimates 600A, 601A with the patient.

In any event, the healthcare professional may select the treatment plan for the patient to follow to achieve the desired result. The selected treatment plan may be transmitted to the patient interface 50 for presentation. The patient may view the selected treatment plan on the patient interface 50. The patient may view on the patient interface 50 the predicted estimate corresponding to the selected treatment plan. In some embodiments, the healthcare professional and the patient may discuss during the telemedicine session the details (e.g., treatment protocol using treatment apparatus 70, diet regimen, medication regimen, etc.) in real-time or in near real-time. In some embodiments, the server 30 may control, based on the selected treatment plan and during the telemedicine session, the treatment apparatus 70 as the user uses the treatment apparatus 70.

It may be useful and encouraging to a patient to provide the patient with information about an incentive such as an awardable product, object, activity, accolade, title, discount, coupon, other incentive, or some combination of these, and to communicate to the patient that the patient could earn the incentive by adhering to the selected treatment plan or achieving a certain percentage or degree of adherence thereto, and this meaning of “adherence” shall also apply herein with respect to the use of that term. The patient may be provided with this information via patient interface 50. If the form of the incentive itself is such that it may be provided via the patient interface 50, the patient may be provided with the incentive once it has been confirmed that the patient has adhered to the treatment plan. For example, if the incentive is an awardable pack of movie tickets, the patient may be provided with electronic codes or links to redeem the movie tickets via the patient interface 50.

In some embodiments, patients may be offered the same incentive. However, in other embodiments, different patients or the same patients at different times may be offered different incentives. A particular incentive for a particular patient may be selected based on the predicted estimate of time. For example, a higher-value incentive may be offered to patients whose treatment plan requires greater and/or prolonged effort(s), than to those patients whose treatment plan requires less or less prolonged effort(s). The offering of an incentive may be intended to ensure that the patient remains diligent about adhering to the treatment plan. In certain cases, it may be determined that there is no utility in offering a particular patient an incentive.

A particular incentive offered to a particular patient may be selected from a set of alternative incentives based on the attributes of the patient, subject to laws and regulations of the relevant jurisdictions, regulatory agencies, and the like, and further subject to relevancy based on geography, location, and the like. A set of multiple selectable incentives may be offered to a particular patient, from which the patient can select their choice of one or a subset. For example, a particular patient being offered one of multiple alternative incentives such as movie tickets, sports event discounts, and product specials may be enabled to choose one or another based on their own differential preferences for different kinds of entertainment. Alternatively, some or all incentives available to be offered may be presented to the patient so that the patient is not being offered a limited subset of the kinds of incentives, thereby enabling the patient to self-select their incentive.

An incentive or set of incentives offered for selection by a patient may be selected using the artificial intelligence engine based on the cohort in which the patient has been included (or cohorts, as in alternative embodiments, a one-to-many relationship may exist in which a patient is associated with more than one cohort). For example, it may be determined using the artificial intelligence engine that individuals in a first cohort tend to be successful in adhering to their treatment plans when offered one kind or instance of an incentive, whereas individuals in a second cohort tend to be successful in adhering to their treatment plans when offered a different kind or instance of an incentive. A patient determined to be in the first cohort may not be offered any of the kinds or instances of incentives that would be offered to a patient determined to be in the second cohort. Alternatively, a patient determined to be in the first cohort may be offered the kinds or instances of incentives for their cohort first, while being provided with an option to receive offers of kinds or instances of incentives for another cohort in the event the patient was not interested in the kinds or instances of incentives first offered. As such, using the artificial intelligence engine for selecting incentives or sets of incentives or kinds of incentives to offer a patient may be not for the purpose of excluding certain incentives or sets of incentives or kinds of incentives from being offered to a patient, but to establish an order or format in which the incentives could be presented to the patient for browsing and selection.

The patient interface 50 may display information about the incentive after it has been selected by a patient as, for example, during performance of the treatment plan. The patient interface 50, during treatment, may display a message stating “Congratulations! You have gotten through 75% of the predicted time for your treatment and you have done a great job in meeting the goals of your treatment plan. You'll be able to return to work soon, so to celebrate, you've earned free movie tickets!”

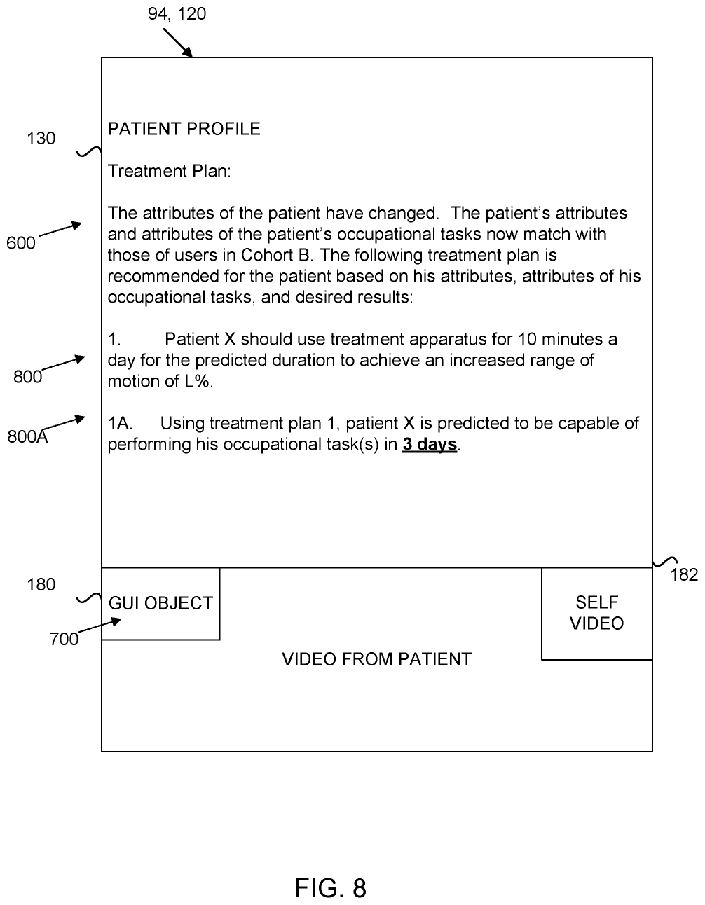

FIG. 8 shows an embodiment of the overview display 120 of the healthcare professional interface 94 presenting, in real-time during a telemedicine session, recommended treatment plans that have changed as a result of patient data changing according to the present disclosure. As may be appreciated, the treatment apparatus 70 and/or any computing device (e.g., patient interface 50) may transmit data while the patient uses the treatment apparatus 70 to perform a treatment plan. The data may include updated attributes of the patient. For example, the updated attributes may include new performance information and/or measurement information. The performance information may include a speed of a portion of the treatment apparatus 70, a range of motion achieved by the patient, a force exerted on a portion of the treatment apparatus 70, a heartrate of the patient, a blood pressure of the patient, a respiratory rate of the patient, and so forth.

In one embodiment, the data received at the server 30 may be input into the trained machine learning model 13, which may determine that the attributes indicate the patient is on track for the current treatment plan. Determining the patient is on track for the current treatment plan may cause the trained machine learning model 13 to adjust a parameter of the treatment apparatus 70. The adjustment may be based on a next step of the treatment plan to further improve the performance of the patient.

In one embodiment, the data received at the server 30 may be input into the trained machine learning model 13, which may determine that the attributes indicate the patient is not on track (e.g., behind schedule, not able to maintain a speed, not able to achieve a certain range of motion, is in too much pain, etc.) for the current treatment plan or is ahead of schedule (e.g., exceeding a certain speed, exercising longer than specified with no pain, exerting more than a specified force, etc.) for the current treatment plan. The trained machine learning model 13 may determine that the attributes of the patient no longer match with the attributes of the patients in the cohort to which the patient is assigned. Accordingly, the trained machine learning model 13 may reassign the patient to another cohort that includes qualifying the patient's attributes. As such, the trained machine learning model 13 may select a new treatment plan, and may predict a second estimate of when the patient performing the new treatment plan would be capable of performing the occupational task(s) associated with the patient. Control of the treatment apparatus 70 would thereafter be based on the new treatment plan.

In some embodiments, prior to controlling the treatment apparatus 70, the server 30 may provide the new treatment plan 800, along with a new estimate 800A, to the healthcare professional interface 94 for presentation in the patient profile 130. As depicted, the patient profile 130 indicates “The attributes of the patient have changed. The patient's attributes and attributes of the patient's occupational tasks now match with those of users in Cohort B. The following treatment plan is recommended for the patient based on his attributes, attributes of his occupational tasks, and desired results.” The patient profile 130 then contains and presents the new treatment plan 800 (“Patient X should use treatment apparatus for 10 minutes a day for the predicted duration to achieve an increased range of motion of L %”) and the new estimate 800A (“Using treatment plan 1, patient X is predicted to be capable of performing his occupational task(s) in 3 days.”). The healthcare professional (healthcare professional) may select the new treatment plan 800, and the server 30 may receive the selection. The server 30 may control the treatment apparatus 70 based on the new treatment plan 800. In some embodiments, the new treatment plan 800 may be transmitted to the patient interface 50 such that the patient may view the details of the new treatment plan 800.

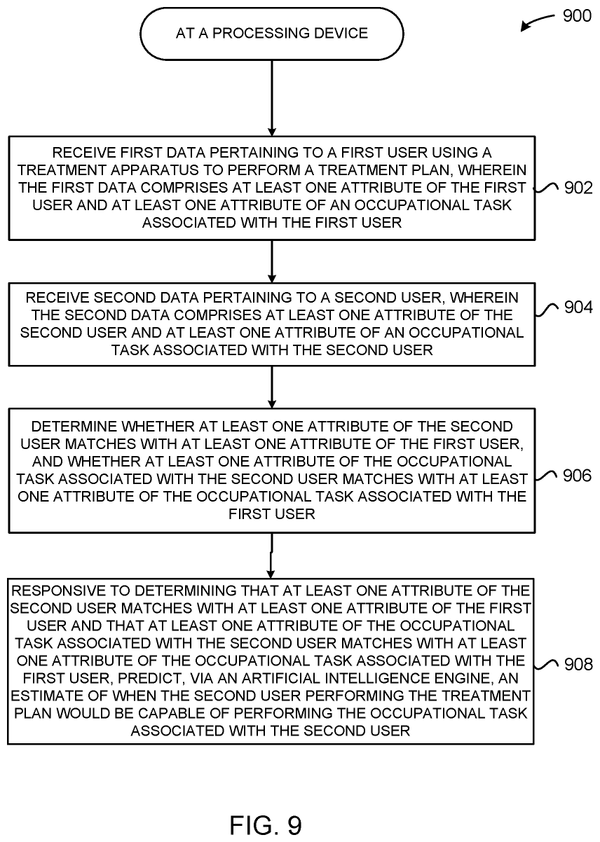

FIG. 9 shows an example embodiment of a method 900 for predicting an estimate of when a patient would be capable of performing an occupational task. The method 900 is performed by processing logic that may include hardware (circuitry, dedicated logic, etc.), software (such as is run on a general-purpose computer system or a dedicated machine), or a combination of both. The method 900 and/or each of its individual functions, routines, subroutines, or operations may be performed by one or more processors of a computing device (e.g., any component of FIG. 1, such as server 30 executing the artificial intelligence engine 11). In certain implementations, the method 900 may be performed by a single processing thread. Alternatively, the method 900 may be performed by two or more processing threads, each thread implementing one or more individual functions, routines, subroutines, object-oriented method, or operations of the object-oriented methods.

For simplicity of explanation, the method 900 is depicted and described as a series of operations. However, operations in accordance with this disclosure can occur in various orders and/or concurrently, and/or with other operations not presented and described herein. For example, the operations depicted in the method 900 may occur in combination with any other operation of any other method disclosed herein. Furthermore, not all illustrated operations may be required to implement the method 900 in accordance with the disclosed subject matter. In addition, those skilled in the art will understand and appreciate that the method 900 could alternatively be represented as a series of interrelated states via a state diagram or events.

At 902, the processing device may receive first data pertaining to a first user using a treatment apparatus 70 to perform a treatment plan. The first data may include at least one attribute of the first user and at least one attribute of an occupational task associated with the first user.

At 904, the processing device may receive second data pertaining to a second user. The second data may include at least one attribute of the second user and at least one attribute of an occupational task associated with the second user.

The attributes of the first user and the second user may include personal information, performance information, measurement information, or some combination thereof. In some embodiments, the personal information may include an age, a weight, a gender, a height, a body mass index, a medical condition, a familial medication history, an injury, or a medical procedure. In some embodiments, the performance information may include an elapsed time of using the treatment apparatus, an amount of force exerted on a portion of the treatment apparatus, a range of motion achieved on the treatment apparatus, a movement speed of a portion of the treatment apparatus, an indication of a set of pain levels using the treatment apparatus, or some combination thereof. In some embodiments, the measurement information may include a vital sign, a respiration rate, a heartrate, a temperature, or some combination thereof.

The attributes of an occupational task of the first user and the second user may include a stamina requirement, a strength requirement, a flexibility requirement, a pliability requirement, a range of motion requirement, a sustained attention requirement, a speech requirement, a height requirement, a weight requirement, a limb use requirement, or some combination thereof.

At 906, the processing device may determine whether at least one attribute of the second user matches with at least one attribute of the first user, and whether at least one attribute of the occupational task associated with the second user matches with at least one attribute of the occupational task associated with the first user.

At 908, responsive to determining that at least one attribute of the second user matches with at least one attribute of the first user and that at least one attribute of the occupational task associated with the second user matches with at least one attribute of the occupational task associated with the first user, the processing device may predict, via an artificial intelligence engine, an estimate of when the second user performing the treatment plan would be capable of performing the occupational task associated with the second user.

The predicting may include predicting an estimate of when a readiness score of the second user performing the treatment plan would satisfy a readiness score threshold for the occupational task associated with the second user. A readiness score for the second user may be based on at least one capability score of the second user, where the capability score represents a measure of the capability of the second user to perform the occupational task. The occupational task may be associated with a required capability score or, without limitation, with a range of capability scores or with a threshold capability score. The second user's capability score may be a measure of that second user's capability to perform the occupational task as measured against whichever requirements are associated with the performance of the occupational tasks, and where those requirements may be represented by, without limitation, a required capability score, a range of capability scores or a threshold capability score.

A capability score may be a stamina score, a strength score, a flexibility score, a pliability score, a range of motion score, a sustained attention score, a speech score, a height score, a weight score, a limb use score, or some combination thereof. Similarly, a readiness score threshold may be based on at least one capability score threshold. A capability score threshold may be a stamina score threshold, a strength score threshold, a flexibility score threshold, a pliability score threshold, a range of motion score threshold, a sustained attention score threshold, a speech score threshold, a height score threshold, a weight score threshold, a limb use score threshold, or some combination thereof. It will be appreciated that, while a number of capability scores for a person may be available, a particular occupational task may not require all of the capabilities that a person may possess such that only a subset of the capability scores of a person may be required to determine whether the person's readiness score satisfies a readiness score threshold for the occupational task. Alternatively, a derived score based on one or more capability scores may itself be used as a capability score; that is, a capability score may be for a particular capability or it may be derived or computed based on other capability scores, including but not limited to, other derived capability scores.

A readiness score threshold may include multiple components, and a readiness score may include multiple corresponding components. Determining whether the readiness score of a person satisfies the readiness score threshold of the occupational task may include determining whether each capability score threshold comprising the readiness score threshold is satisfied by the person's respective capability score. For example, a readiness score threshold for a particular occupational task of lifting boxes off a truck in a warehouse may be composed of multiple individual capability score thresholds, including a strength score threshold, a stamina score threshold, and a flexibility score threshold. In this example, the person's readiness score may be composed of multiple corresponding individual capability scores including a strength score, a stamina score, and a flexibility score. Therefore, for the person's readiness score to satisfy the readiness score threshold for this particular occupational task, each of the capability scores must satisfy the corresponding capability score thresholds. In this occupational task example, therefore, the person's strength score must satisfy the strength score threshold, the person's stamina score must satisfy the stamina score threshold, and the person's flexibility score must satisfy the flexibility score threshold. If the person's stamina score satisfies the stamina score threshold, the person's strength score satisfies the strength score threshold, and the person's flexibility score satisfies the flexibility score threshold, then the readiness score of the person shall be deemed to satisfy the readiness score threshold of the occupational task, and the person shall be deemed to be capable of performing the occupational task. In this example, if any of the capability scores does not satisfy its respective capability score threshold, then the readiness score shall not be deemed to satisfy the respective readiness score threshold.

Table 1 sets out an example set of capability score thresholds for a given occupational task.

TABLE 1

| | Capability Score Threshold |
|---|---|
| Strength | 5 |
| Stamina | 8 |
| Flexibility | 6 |

In this example, a person's scores in strength, stamina and flexibility are required to determine whether the person is capable of performing the occupational task. A person's other capability scores are not required to be used to determine if the person is capable of performing the occupational task. In this example, the strength score threshold (out of, for example, 10) is 5, the stamina score threshold is 8, and the flexibility score threshold is 6. The readiness score threshold may be expressed as 5-8-6. If the readiness score of a person—also expressed in a similar manner—satisfies the readiness score threshold, then the person shall be deemed to be capable of performing the occupational task. In this example, the readiness score of a person satisfies the readiness score threshold for the occupational task if each of the capability scores satisfies respective capability score thresholds. Table 2 sets out an example set of capability scores for a person at a Time A, such as a time at which the person is first assessed for capabilities.

TABLE 2

| | Capability Score |
|---|---|
| Strength | 4 |
| Stamina | 8 |
| Flexibility | 4 |

In Table 2, the person's strength score is 4, his stamina score is 8, and his flexibility score is 4, so that for a multiple-component readiness score, this may be expressed as 4-8-4. In this example, since the strength score of 4 is less than, and thus does not satisfy, the strength score threshold of 5, this is a sufficient condition for the readiness score threshold to be determined not to have been satisfied, such that the person would accordingly be deemed not capable of performing the occupational task. Note that another sufficient condition for the readiness score threshold to be determined not to have been satisfied in this example would be if the flexibility score of 4 were less than, and thus would not satisfy, the flexibility score threshold of 6.

Table 3 sets out an example set of capability scores for the person at a Time B, such as a time after the person has been performing a treatment plan.

TABLE 3

| | Capability Score |
|---|---|
| Strength | 5 |
| Stamina | 9 |
| Flexibility | 6 |

In Table 3, the person's strength score is 5, his stamina score is 9, and his flexibility score is 6, for a readiness score that may be expressed as 5-9-6. In this example, since all the capability scores pertaining to the occupational task are greater than or equal to respective capability score thresholds for the occupational task, the readiness score is deemed to have satisfied the readiness score threshold and the person may be deemed to be capable of performing the occupational task.

It will be appreciated that, in some embodiments, a readiness score threshold may include only a single component corresponding to a single capability score threshold, and a readiness score may include only a single component corresponding to a single capability score. A single component of a readiness score threshold may be, simply, a single capability score threshold required by a particular occupational task, and the single component of a readiness score of a person with respect to the occupational task may be simply a single corresponding capability score of the person. For example, a particular occupational task may require only one capability such as flexibility, and a certain flexibility score to meet a certain flexibility score threshold, such that the readiness score threshold could be equivalent to, or derived from, a flexibility score threshold for the occupational task, and a readiness score for a person corresponding to the occupational task could be equivalent to, or derived from, a flexibility score of the person.

In some embodiments, a readiness score threshold may include a single component determined as a weighted average of multiple capability score thresholds, and a readiness score may include a single component determined as a weighted average of multiple capability scores of a person. For example, a readiness score threshold for the particular occupational task of lifting boxes off a truck in a warehouse may be composed of a weighted average of individual capability score thresholds including a strength score threshold, a stamina score threshold, and a flexibility score threshold. It may be determined by an employer, a health and safety regulatory body, or some other entity responsible for setting standards for occupational tasks that, for this particular occupational task, while stamina, strength and flexibility are required, stamina is more important than strength and flexibility. A readiness score threshold therefore may be determined as a weighted average that lends to a stamina score threshold a higher weighting in the weighted average calculation than to each of the strength score threshold and flexibility score threshold. For example, a stamina score threshold may be assigned 50% weighting, while each of the strength score threshold and flexibility score thresholds may be assigned a 25% weighting (or 50-30-20 or 65-15-20 or 40-35-25 or any other set of weightings such that the total weight is equal to 100, here and in all other such examples set forth in this disclosure), during calculation of the averages of these for determining the readiness score threshold. Similarly, in this example, the readiness score of a person may be determined as a weighted average that assigns to a stamina score the higher weight than to each of the strength score and flexibility score. For example, to correspond to the weighted average calculation of the readiness score threshold in this example, for a weighted average calculation of the person's readiness score, his stamina score may be assigned a 50% weighting, while each of his strength score and flexibility score may be assigned a 25% weighting (or any other set of weights such that the total weight is equal to 100). Then, once the resultant readiness score of the person satisfies the resultant readiness score threshold of the occupational task, the person may be deemed to be capable of performing the occupational task. Note that, due to the weighted averaging, a readiness score may satisfy a readiness score threshold even if a particular capability score does not satisfy its corresponding particular capability score threshold for the occupational task. For example, a person's strength score may not quite satisfy the corresponding strength score for the occupational task, but due to the person's stamina score and flexibility score and the respective differential weightings applied to each of the strength score, stamina score and flexibility score, the resultant readiness score being calculated as a weighted average may nevertheless satisfy the corresponding readiness score threshold such that the person may be deemed to be capable of performing the occupational task.

In such an example, the person may have a flexibility score that is lower than the flexibility score threshold of the occupational task, but due to differential weightings and that person's stamina score exceeding the stamina score threshold of the occupational task, the person may be deemed ready to perform the occupational task.

Table 4 sets out an example set of capability score thresholds for a given occupational task.

TABLE 4

| | Capability Score Threshold | Weight |
|---|---|---|
| Strength | 5 | 0.25 |
| Stamina | 8 | 0.5 |
| Flexibility | 6 | 0.25 |

In this example, a person's capability scores for strength, stamina and flexibility may be required to determine if the person is capable of performing the occupational task. A person's other capability scores (such as a height score) may not be needed to determine if the person is capable of performing the occupational task. In this example, in the readiness score threshold calculation, the strength score threshold of 5 is given a weight of 0.25, the stamina score threshold of 8 is given a weight of 0.5, and the flexibility score threshold of 6 is given a weight of 0.25, for a single component readiness score threshold of 6.75. Similarly, in a readiness score calculation for a person, the person's strength score is given a weight of 0.25, his stamina score is given a weight of 0.5, and his flexibility score is given a weight of 0.25. If the readiness score of a person—calculated as a weighted average of the person's capability scores for each of strength, stamina, and flexibility—satisfies the readiness score threshold, then the person shall be deemed to be capable of performing the occupational task. Table 5 sets out an example set of capability scores for a person at a Time A, such as a time at which the person is first assessed for capabilities.

TABLE 5

| | Capability Score | Weight |
|---|---|---|
| Strength | 4 | 0.25 |
| Stamina | 8 | 0.5 |
| Flexibility | 4 | 0.25 |

In Table 5, the person's strength score is 4, his stamina score is 8, and his flexibility score is 4, for a single component readiness score—calculated as a weighted average according to the weights shown in Table 5—of 6.0. In this example, since the readiness score of 6.0 is not equal to or greater than the readiness score threshold of 6.75, the readiness score does not satisfy the readiness score threshold and the person is deemed to be not yet capable of performing the occupational task.

Table 6 sets out an example set of capability scores for the person at a Time B, such as a time after the person has been performing a treatment plan.

TABLE 6

| | Capability Score | Weight |
|---|---|---|
| Strength | 5 | 0.25 |
| Stamina | 9 | 0.5 |
| Flexibility | 5 | 0.25 |

In Table 6, the person's strength score is 5, his stamina score is 9, and his flexibility score is 5, for a single component readiness score—calculated as a weighted average according to the weights shown in Table 6—of 7.0. In this example, since the single component readiness score of 7.0 is greater than the readiness score threshold of 6.75, the readiness score satisfies the readiness score threshold and the person may be deemed capable of performing the occupational task.

The treatment plan may include a treatment protocol that specifies using the treatment apparatus 70 to perform certain exercises for certain lengths of time and a periodicity for performing the exercises. The treatment protocol may also specify parameters of the treatment apparatus 70 for each of the exercises. For example, a two-week treatment protocol for a person having certain attributes (e.g., respiration, weight, age, injury, current range of motion, heartrate, etc.) may specify the exercises for a first week and a second week. The exercise for the first week may include pedaling a bicycle for a 10-minute time period where the pedals gradually increase or decrease a range of motion every 1 minute throughout the 10-minute time period. The exercise for the second week may include pedaling a bicycle for a 5-minute time period where the pedals aggressively increase or decrease a range of motion every 1 minute throughout the 10-minute time period.

In some embodiments, the processing device may control, based on the treatment plan, the treatment apparatus 70 while the second user uses the treatment apparatus. In some embodiments, the controlling may be performed by the server 30 distal from the treatment apparatus 70 (e.g., during a telemedicine session). Controlling the treatment apparatus 70 distally may include the server 30 transmitting, based on the treatment plan, a control instruction to change a parameter of the treatment apparatus 70 at a particular time to increase a likelihood of a positive effect of continuing to use the treatment apparatus or to decrease a likelihood of a negative effect of continuing to use the treatment apparatus. For example, the treatment plan may include information (based on historical information of people having certain attributes and performing exercises in the treatment plan) indicating there may be diminishing returns after a certain amount of time of performing a certain exercise. Accordingly, the server 30, executing one or more machine learning models 13, may transmit a control signal to the treatment apparatus 70 to cause the treatment apparatus 70 to change a parameter (e.g., slow down, stop, etc.).

In some embodiments, the treatment apparatus used by the first user may be the treatment apparatus used by the second user, or the treatment apparatus used by the first user may not be the treatment apparatus used by the second user. For example, if the first user and the second user are members of a family, then they may both use one treatment apparatus at different times. If the first user and the second user live in different residences, then the first user and the second user may use different treatment apparatuses, either simultaneously or at different times.

The term “same” used in connection with two or more treatment apparatuses may refer to the two or more treatment apparatuses being distinct physical objects but having the same specifications, such as being the same model of treatment apparatus.

In some embodiments, the processing device may continue to receive data while the second user uses the treatment apparatus 70 to perform the treatment plan. The data received may include attributes of the second user while the second user uses the treatment apparatus 70 to perform the treatment plan. The attributes may include information pertaining to measurements (e.g., respiration, heartrate, temperature, perspiration) and performance (e.g., range of motion, force exerted on a portion of the treatment apparatus 70, speed of actuating a portion of the treatment apparatus 70, etc.). The data may indicate that the second user is improving (e.g., maintaining a desired speed of the treatment plan, range of motion, and/or force) as expected in view of the treatment plan for a person having similar data. Accordingly, the processing device may adjust, via a trained machine learning model 13, based on the data and the treatment plan, a parameter of the treatment apparatus 70. For example, the data may indicate the second user is pedaling a portion of the treatment apparatus 70 for 3 minutes at a certain speed. Thus, the machine learning model may adjust, based on the data and the treatment plan, an amount of resistance of the pedals to attempt to cause the second user to achieve a certain result (e.g., strengthen one or more muscles). The certain result may have been achieved by other users with similar data (e.g., attributes including performance, measurements, etc.) exhibited by the second user at a particular point in a treatment plan.

In some embodiments, the processing device may receive, from the treatment apparatus 70, data pertaining to second attributes of the second user while the second user uses the treatment apparatus 70 to perform the treatment plan. The second attributes may include information pertaining to measurements (e.g., respiration, heartrate, temperature, perspiration) and performance (e.g., range of motion, force exerted on a portion of the treatment apparatus 70, speed of actuating a portion of the treatment apparatus 70, etc.) of the second user as the second user uses the treatment apparatus 70 to perform the treatment plan. In some embodiments, the processing device may determine, based on the attributes, that the second user is improving faster than expected for the treatment plan or is not improving (e.g., unable to maintain a desired speed of the treatment plan, range of motion, and/or force) as expected for the treatment plan.

The processing device may determine that the second attributes of the second user match with attributes of a third user assigned to a second cohort. The second cohort may include data for people having different attributes than the cohort to which the second user was initially assigned. Responsive to determining the second attributes of the second user match with the attributes of the third user and that attributes of the occupational tasks of the second user match with attributes of the occupational tasks of the third user, the processing device may assign the second user to the second cohort and select, via the trained machine learning model, a second treatment plan for the second user. Accordingly, the treatment plan for a user using the treatment apparatus 70 may be dynamically adjusted, in real-time while the user is using the treatment apparatus 70, to best fit the attributes of the second user and enhance a likelihood the second user achieves a desired result experienced by other people in a particular cohort to which the second user is assigned. The second treatment plan may have been performed by the third user with similar attributes to the second user and occupational tasks similar to those of the second user, and as a result of performing the second treatment plan, the third user may have achieved a desired result. The processing device may control, based on the second treatment plan, the treatment apparatus 70 while the second user uses the treatment apparatus.

The processing device may determine that the second attributes of the second user match with attributes of a third user assigned to a second cohort. The second cohort may include data for people having different attributes than the cohort to which the second user was initially assigned. Responsive to determining the second attributes of the second user match with the attributes of the third user and that attributes of the occupational tasks of the second user match with attributes of the occupational tasks of the third user, the processing device may assign the second user to the second cohort and select, via the trained machine learning model, the same treatment plan for the second user but with a prediction of a second estimate of when the second user performing the treatment plan would be capable of performing the occupational tasks associated with the second user. The same treatment plan may have been performed by the third user, wherein the third user has attributes similar to the second user's and occupational tasks similar to the second user's, and as a result of performing the same treatment plan, the third user may have achieved a desired result but in a different time frame that had been originally predicted for the second user, thus making a second estimate prediction preferable. Data regarding the prediction of the second estimate may be provided via the healthcare professional interface 94, 120 to a user such as a healthcare professional. The processing device may continue to control, based on the treatment plan, the treatment apparatus 70 while the second user uses the treatment apparatus.

In some embodiments, responsive to determining the attributes of the second user do not match with the attributes of the first user and/or that attributes of the occupational tasks of the second user do not match with attributes of the occupational tasks of the first user, the processing device may determine whether at least the attributes of the second user match with attributes of a third user and attributes of the occupational tasks associated with the second user match with attributes of the occupational tasks associated with the third user, the third user having been assigned to a second cohort. Responsive to determining the attributes of the second user match with the attributes of the third user and that the attributes of the occupational tasks of the second user match with the attributes of the occupational tasks of the third user, the processing device may assign the second user to the second cohort and select, via the trained machine learning model, a second treatment plan for the second user. The processing device may also predict, via the trained machine learning model, a second estimate of when the patient performing the second treatment plan will be capable of performing the occupational tasks associated with the second user. The second treatment plan may have been performed by the third user with similar attributes to the second user and with attributes of occupational tasks similar to the third user's, and as a result of performing the second treatment plan, the third user may have achieved a desired result and in a different or the same time frame that had been originally predicted for the second user, thus making a second estimate prediction preferable. Data regarding the selected second treatment plan and the prediction of the second estimate may be provided to a user such as a healthcare professional via the healthcare professional interface 94, 120, and the healthcare professional may select or decline the second treatment plan. In the event the healthcare professional selects the second treatment plan via the healthcare professional interface 94, 120, the processing device may control, based on the second treatment plan, the treatment apparatus 70 while the second user uses the treatment apparatus. Rather than automatically assigning the second user to the second cohort, assigning the second user to the second cohort may be done only in the event that the healthcare professional selects the second treatment plan, and is not done if the healthcare professional declines the second treatment plan.

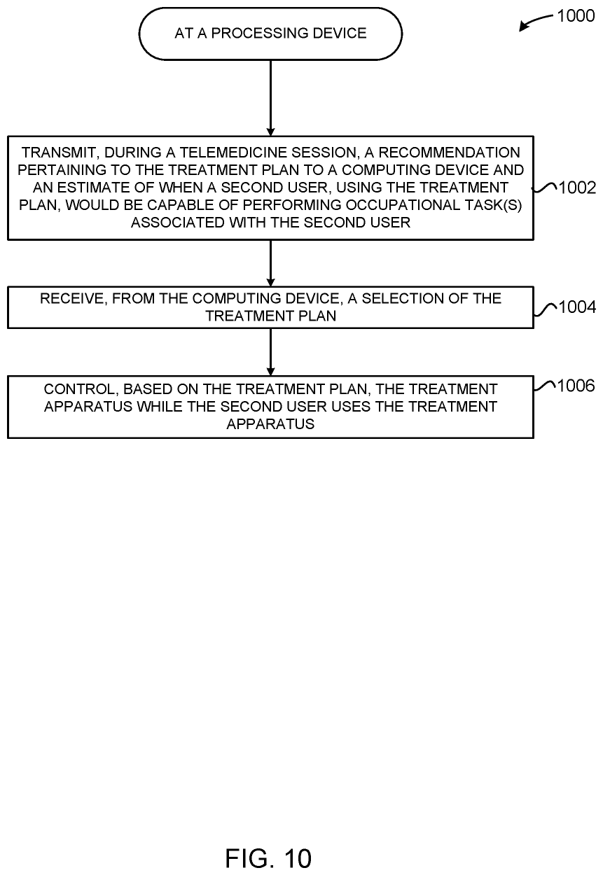

FIG. 10 shows an example embodiment of a method 1000 for presenting, during a telemedicine session, the recommended treatment plan to a healthcare professional according to the present disclosure. Method 1000 includes operations performed by processors of a computing device (e.g., any component of FIG. 1, such as server 30 executing the artificial intelligence engine 11). In some embodiments, one or more operations of the method 1000 are implemented in computer instructions stored on a memory device and executed by a processing device. The method 1000 may be performed in the same or a similar manner as described above in regard to method 900. The operations of the method 1000 may be performed in some combination with any of the operations of any of the methods described herein.

In some embodiments, the method 1000 may occur after 908 in the method 900 depicted in FIG. 9 and prior to controlling the treatment apparatus 70. That is, the method 1000 may occur prior to the server 30 executing the one or more machine learning models 13 controlling the treatment apparatus 70.

Regarding the method 1000, at 1002, prior to controlling the treatment apparatus 70 while the second user uses the treatment apparatus 70, the processing device may provide, during a telemedicine or telehealth session, a recommendation pertaining to the treatment plan to a computing device (e.g., healthcare professional interface 94) of a healthcare professional, as well as an estimate of when the second user performing the treatment plan would be capable of performing occupational task(s) associated with the second user. The recommendation and estimate may be presented on a display screen of the computing device in real-time (e.g., less than 2 seconds) in a portion of the display screen while another portion of the display screen presents video of a user (e.g., patient).

At 1004, the processing device may receive, from the computing device of the healthcare professional, a selection of the treatment plan. The healthcare professional may use any suitable input peripheral (e.g., mouse, keyboard, microphone, touchpad, etc.) to select the recommended treatment plan. The computing device may transmit the selection to the processing device of the server 30, which receives the selection. There may any suitable number of treatment plans presented on the display screen. Each of the treatment plans recommended may provide different results and the healthcare professional may consult, during the telemedicine session, with the user to discuss which result the user desires. In some embodiments, the recommended treatment plans and predicted estimates may only be presented on the computing device of the healthcare professional and not on the computing device of the user (patient interface 50). In some embodiments, the healthcare professional may choose an option presented on the healthcare professional interface 94. The option may cause the treatment plans to be transmitted to the patient interface 50 for presentation. The option may cause the corresponding predicted estimates to be transmitted to the patient interface 50 for presentation. In this way, during the telemedicine session, the healthcare professional and the user may view the treatment plans and the predicted estimates at the same time in real-time or in near real-time, which may provide for an enhanced user experience for the user using the computing device. After the selection of the treatment plan is received at the server 30, at 1006, the processing device may control, based on the selected treatment plan, the treatment apparatus while the second user uses the treatment apparatus 70.

Data pertaining to the predicted estimate of when the user performing the selected treatment plan will be capable of performing the occupational task(s) may be made available, via another of the interfaces 20, 90, 92 or 94, to other healthcare professionals or to other personnel. Such other personnel may include administrators of insurance plans or workers' compensation administrators. Such personnel may use the data to plan for upcoming expenditures to be made in respect of the user. The data pertaining to the predicted estimate of time may include data pertaining to a prediction of an estimate of the cost of the user performing the treatment plan to become capable of performing the occupational task(s). The data pertaining to the prediction of an estimate of a cost may be based on a cost-per-day rate for the user and, potentially further on the predicted estimate of time.

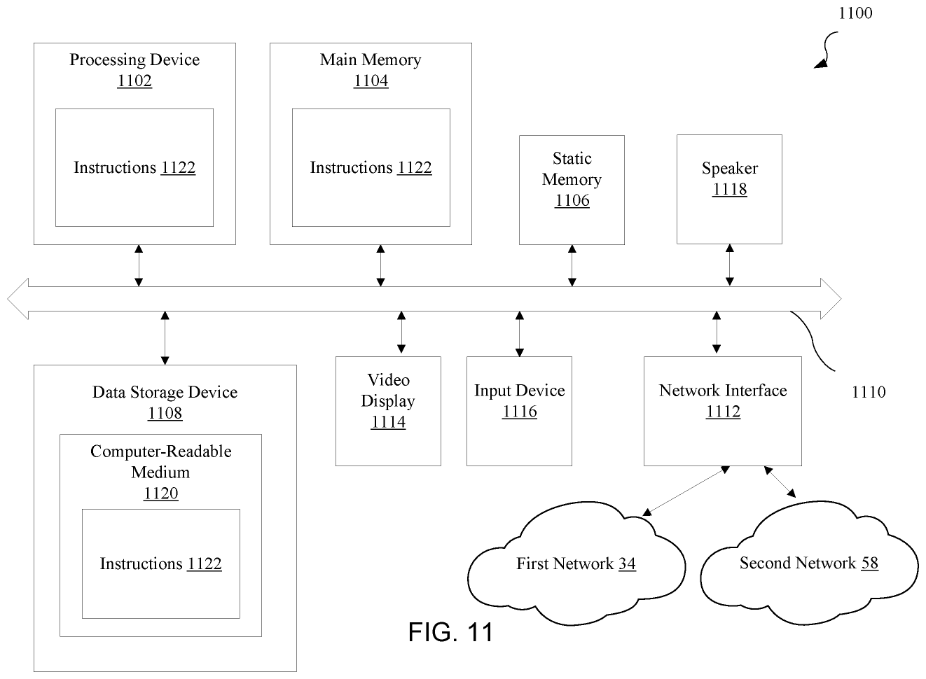

FIG. 11 shows an example computer system 1100 which can perform any one or more of the methods described herein, in accordance with one or more aspects of the present disclosure. In one example, computer system 1100 may include a computing device and correspond to the assistance interface 94, reporting interface 92, supervisory interface 90, clinician interface 20, server 30 (including the AI engine 11), patient interface 50, ambulatory sensor 82, goniometer 84, treatment apparatus 70, pressure sensor 86, or any suitable component of FIG. 1. The computer system 1100 may be capable of executing instructions implementing the one or more machine learning models 13 of the artificial intelligence engine 11 of FIG. 1. The computer system may be connected (e.g., networked) to other computer systems in a LAN, an intranet, an extranet, or the Internet, including via the cloud or a peer-to-peer network. The computer system may operate in the capacity of a server in a client-server network environment. The computer system may be a personal computer (PC), a tablet computer, a wearable (e.g., wristband), a set-top box (STB), a personal Digital Assistant (PDA), a mobile phone, a camera, a video camera, an Internet of Things (IoT) device, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1100 includes a processing device 1102, a main memory 1104 (e.g., read-only memory (ROM), flash memory, solid state drives (SSDs), dynamic random access memory (DRAM) such as synchronous DRAM (SDRAM)), a static memory 1106 (e.g., flash memory, solid state drives (SSDs), static random access memory (SRAM)), and a data storage device 1108, which communicate with each other via a bus 1110.

Processing device 1102 represents one or more general-purpose processing devices such as a microprocessor, central processing unit, or the like. More particularly, the processing device 1102 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1402 may also be one or more special-purpose processing devices such as an application specific integrated circuit (ASIC), a system on a chip, a field programmable gate array (FPGA), a digital signal processor (DSP), network processor, or the like. The processing device 1402 is configured to execute instructions for performing any of the operations and steps discussed herein.

The computer system 1100 may further include a network interface device 1112. The computer system 1100 also may include a video display 1114 (e.g., a liquid crystal display (LCD), a light-emitting diode (LED), an organic light-emitting diode (OLED), a quantum LED, a cathode ray tube (CRT), a shadow mask CRT, an aperture grille CRT, a monochrome CRT), one or more input devices 1116 (e.g., a keyboard and/or a mouse or a gaming-like control), and one or more speakers 1118 (e.g., a speaker). In one illustrative example, the video display 1114 and the input device(s) 1116 may be combined into a single component or device (e.g., an LCD touch screen).

The data storage device 1116 may include a computer-readable medium 1120 on which the instructions 1122 embodying any one or more of the methods, operations, or functions described herein is stored. The instructions 1122 may also reside, completely or at least partially, within the main memory 1104 and/or within the processing device 1102 during execution thereof by the computer system 1100. As such, the main memory 1104 and the processing device 1102 also constitute computer-readable media. The instructions 1122 may further be transmitted or received over a network via the network interface device 1112.

While the computer-readable storage medium 1120 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

### CLAUSES

1. A method comprising:

receiving first data pertaining to a first user using a treatment apparatus to perform a treatment plan, wherein the first data comprises at least one attribute of the first user and at least one attribute of an occupational task associated with the first user;

receiving second data pertaining to a second user, wherein the second data comprises at least one attribute of the second user and at least one attribute of an occupational task associated with the second user;

determining whether at least one attribute of the second user matches with at least one attribute of the first user, and whether at least one attribute of the occupational task associated with the second user matches with at least one attribute of the occupational task associated with the first user; and

responsive to determining that at least one attribute of the second user matches with at least one attribute of the first user and that at least one attribute of the occupational task associated with the second user matches with at least one attribute of the occupational task associated with the first user, predicting, via an artificial intelligence engine, an estimate of when the second user performing the treatment plan would be capable of performing the occupational task associated with the second user.

2. The method of clause 1, wherein the at least one attribute of the first user and of the second user comprises personal information, performance information, measurement information, incentive information, or some combination thereof.

3. The method of clause 2, wherein:

the personal information comprises an age, a weight, a gender, a height, a body mass index, a medical condition, a familial medication history, an injury, a medical procedure, or some combination thereof,

the performance information comprises an elapsed time of using the treatment apparatus, an amount of force exerted on a portion of the treatment apparatus, a range of motion achieved on the treatment apparatus, a movement speed of a portion of the treatment apparatus, an indication of a plurality of pain levels using the treatment apparatus, or some combination thereof,

the measurement information comprises a vital sign, a respiration rate, a heartrate, a temperature, or some combination thereof, and

the incentive information comprises information about an awardable product, object, activity, accolade, title, discount, coupon, or some combination thereof.

4. The method of clause 1, wherein the at least one attribute of an occupational task of the first user and of the second user comprises a stamina requirement, a strength requirement, a flexibility requirement, a pliability requirement, a range of motion requirement, a sustained attention requirement, a speech requirement, a height requirement, a weight requirement, a limb use requirement, or some combination thereof.

5. The method of clause 1, wherein the predicting comprises:

predicting an estimate of when a readiness score of the second user performing the treatment plan would satisfy a readiness score threshold for the occupational task associated with the second user.

6. The method of clause 5, wherein:

the readiness score is based on at least one capability score of the second user; and

the readiness score threshold is based on at least one capability score threshold for the occupational task associated with the second user.

7. The method of clause 6, wherein the at least one capability score is selected from the group consisting of: a stamina score, a strength score, a flexibility score, a pliability score, a range of motion score, a sustained attention score, a speech score, a height score, a weight score, a limb use score, or some combination thereof; and

the at least one capability score threshold is selected from the group consisting of: a stamina score threshold, a strength score threshold, a flexibility score threshold, a pliability score threshold, a range of motion score threshold, a sustained attention score threshold, a speech score threshold, a height score threshold, a weight score threshold, a limb use score threshold, or some combination thereof.

8. The method of clause 6, wherein:

the readiness score is a weighted average of a plurality of capability scores of the second user; and

the readiness score threshold is a weighted average of a plurality of capability score thresholds for the occupational task associated with the second user.

9. The method of clause 1, further comprising:

transmitting at least a portion of the second data, at least a portion of the treatment plan, and data pertaining to the estimate to a computing device of a healthcare professional during a telemedicine session; and

receiving, from the computing device of a healthcare professional, a selection of the treatment plan for the second user.

10. The method of clause 9, wherein the first user is assigned to a first cohort based at least in part on the first data, the method comprising:

responsive to receiving the selection of the treatment plan for the second user from the computing device of the healthcare professional, assigning the second user to the first cohort.

11. The method of clause 10, comprising:

receiving, from the treatment apparatus, third data pertaining to use of the treatment apparatus by the second user performing the treatment plan; and

providing, to a computing device of the healthcare professional, a report pertaining to the third data.

12. The method of clause 11, comprising:

responsive to receiving the third data, predicting, via the artificial intelligence engine, a second estimate of when the second user performing the treatment plan would be capable of performing the occupational task associated with the second user; and

providing, to the computing device of the healthcare professional, a report pertaining to the second estimate.

13. The method of clause 11, wherein the third data pertains to at least one second attribute of the first user while the first user uses the treatment apparatus to perform the treatment plan, the method comprising:

responsive to receiving the third data, determining whether at least one second attribute of the second user matches with at least one attribute of a third user, and whether at least one attribute of the occupational task associated with the second user matches with at least one attribute of an occupational task associated with the third user;

responsive to determining that at least one second attribute of the second user matches with at least one attribute of the third user, and that at least one attribute of the occupational task associated with the second user matches with at least one attribute of an occupational task associated with the third user, predicting, via the artificial intelligence engine, a second estimate of when the second user performing the treatment plan would be capable of performing the occupational task associated with the second user.

14. The method of clause 13, further comprising:

transmitting at least a portion of the third data, at least a portion of the treatment plan, and data pertaining to the second estimate to a computing device of a healthcare professional during a telemedicine session.

15. The method of clause 13, wherein the third user is assigned to a second cohort based at least in part on the third data, the method comprising:

assigning the second user to the second cohort.

16. The method of clause 11, wherein the third data pertains to at least one second attribute of the first user while the first user uses the treatment apparatus to perform the treatment plan, the method comprising:

receiving fourth data pertaining to a third user using the treatment apparatus to perform a second treatment plan;

responsive to receiving the fourth data, determining whether at least one second attribute of the second user matches with at least one attribute of the third user, and whether at least one attribute of the occupational task associated with the second user matches with at least one attribute of an occupational task associated with the third user;

responsive to determining that at least one second attribute of the second user matches with at least one attribute of the third user, and that at least one attribute of the occupational task associated with the second user matches with at least one attribute of an occupational task associated with the third user, predicting, via the artificial intelligence engine, a second estimate of when the second user performing the second treatment plan would be capable of performing the occupational task associated with the second user.

17. The method of clause 16, further comprising:

transmitting at least a portion of the fourth data, at least a portion of the second treatment plan, and the second estimate to a computing device of the healthcare professional during a telemedicine session; and

receiving, from the computing device of a healthcare professional, a selection of the second treatment plan for the second user.

18. The method of clause 17, wherein the third user is assigned to a second cohort based at least in part on the fourth data, the method comprising:

responsive to receiving the selection of the second treatment plan for the second user from the computing device of the healthcare professional, assigning the second user to the second cohort.

19. The method of clause 1, wherein the treatment apparatus used by the first user and the treatment apparatus used by the second user are the same.

20. The method of clause 19, wherein the treatment apparatus used by the first user is the treatment apparatus used by the second user.

21. The method of clause 9, wherein the data pertaining to the estimate comprises data pertaining to a prediction of an estimate of a cost of the first user becoming capable of performing the occupational task associated with the second user.

22. The method of clause 1, wherein the artificial intelligence engine trains a machine learning model, using at least the first data, to compare in real-time the second data to a plurality of data and to predict the estimate.

23. The method of clause 1, wherein the artificial intelligence engine trains the machine learning model, using at least the first data, to compare in real-time the second data to the plurality of data and to select at least one awardable incentive to offer to the second user for adhering to the treatment plan.

24. The method of clause 1, further comprising controlling, based on the treatment plan and while the second user uses the treatment apparatus, the treatment apparatus.

25. The method of clause 24, further comprising:

prior to controlling the treatment apparatus while the second user uses the treatment apparatus, providing to a computing device of a healthcare professional, during a telemedicine session, a recommendation pertaining to the treatment plan;

receiving, from the computing device of the healthcare professional, at least a portion of the treatment plan; and

controlling, based on the at least a portion of the treatment plan and while the second user uses the treatment apparatus, the treatment apparatus.

26. The method of clause 24, wherein the controlling is performed by a server distal from the treatment apparatus.

27. A tangible, non-transitory computer-readable medium storing instructions that, when executed, cause a processing device to:

receive first data pertaining to a first user using a treatment apparatus to perform a treatment plan, wherein the first data comprises at least one attribute of the first user and at least one attribute of an occupational task associated with the first user;

receive second data pertaining to a second user, wherein the second data comprises at least one attribute of the second user and at least one attribute of an occupational task associated with the second user;

determine whether at least one attribute of the second user matches with at least one attribute of the first user, and whether at least one attribute of the occupational task associated with the second user matches with at least one attribute of the occupational task associated with the first user; and

responsive to determining that at least one attribute of the second user matches with at least one attribute of the first user and that at least one attribute of the occupational task associated with the second user matches with at least one attribute of the occupational task associated with the first user, predict, via an artificial intelligence engine, an estimate of when the second user performing the treatment plan would be capable of performing the occupational task associated with the second user.

28. The tangible, non-transitory computer-readable medium of clause 27, wherein the at least one attribute of the first user and of the second user comprises personal information, performance information, measurement information, incentive information, or some combination thereof.

29. The tangible, non-transitory computer-readable medium of clause 28, wherein:

the personal information comprises an age, a weight, a gender, a height, a body mass index, a medical condition, a familial medication history, an injury, a medical procedure, or some combination thereof,

the performance information comprises an elapsed time of using the treatment apparatus, an amount of force exerted on a portion of the treatment apparatus, a range of motion achieved on the treatment apparatus, a movement speed of a portion of the treatment apparatus, an indication of a plurality of pain levels using the treatment apparatus, or some combination thereof,

the measurement information comprises a vital sign, a respiration rate, a heartrate, a temperature, or some combination thereof, and

the incentive information comprises information about an awardable product, object, activity, accolade, title, discount, coupon, or some combination thereof.

30. The tangible, non-transitory computer-readable medium of clause 27, wherein the at least one attribute of an occupational task of the first user and of the second user comprises a stamina requirement, a strength requirement, a flexibility requirement, a pliability requirement, a range of motion requirement, a sustained attention requirement, a speech requirement, a height requirement, a weight requirement, a limb use requirement, or some combination thereof.

31. The tangible, non-transitory computer-readable medium of clause 27, wherein to predict an estimate of when the second user performing the treatment plan would be capable of performing the occupational task associated with the second user, the instructions, when executed, cause the processing device to:

predict an estimate of when a readiness score of the second user performing the treatment plan would satisfy a readiness score threshold for the occupational task associated with the second user.

32. The tangible, non-transitory computer-readable medium of clause 31, wherein:

the readiness score is based on at least one capability score of the second user; and

the readiness score threshold is based on at least one capability score threshold for the occupational task associated with the second user.

33. The tangible, non-transitory computer-readable medium of clause 32, wherein the at least one capability score is selected from the group consisting of: a stamina score, a strength score, a flexibility score, a pliability score, a range of motion score, a sustained attention score, a speech score, a height score, a weight score, a limb use score, or some combination thereof; and

the at least one capability score threshold is selected from the group consisting of: a stamina score threshold, a strength score threshold, a flexibility score threshold, a pliability score threshold, a range of motion score threshold, a sustained attention score threshold, a speech score threshold, a height score threshold, a weight score threshold, a limb use score threshold, or some combination thereof.

34. The tangible, non-transitory computer-readable medium of clause 32, wherein:

the readiness score is a weighted average of a plurality of capability scores of the second user; and

the readiness score threshold is a weighted average of a plurality of capability score thresholds for the occupational task associated with the second user.

35. The tangible, non-transitory computer-readable medium of clause 27, wherein the instructions, when executed, cause the processing device to:

transmit at least a portion of the second data, at least a portion of the treatment plan, and data pertaining to the estimate to a computing device of a healthcare professional during a telemedicine session; and

receive, from the computing device of a healthcare professional, a selection of the treatment plan for the second user.

36. The tangible, non-transitory computer-readable medium of clause 35, wherein the first user is assigned to a first cohort based at least in part on the first data, wherein the instructions, when executed, cause the processing device to:

responsive to receiving the selection of the treatment plan for the second user from the computing device of the healthcare professional, assign the second user to the first cohort.

37. The tangible, non-transitory computer-readable medium of clause 36, wherein the instructions, when executed, cause the processing device to:

receive, from the treatment apparatus, third data pertaining to use of the treatment apparatus by the second user performing the treatment plan; and

provide, to a computing device of the healthcare professional, a report pertaining to the third data.

38. The tangible, non-transitory computer-readable medium of clause 37, wherein the instructions, when executed, cause the processing device to:

responsive to receiving the third data, predict, via the artificial intelligence engine, a second estimate of when the second user performing the treatment plan would be capable of performing the occupational task associated with the second user; and

provide, to the computing device of the healthcare professional, a report pertaining to the second estimate.

39. The tangible, non-transitory computer-readable medium of clause 37, wherein the third data pertains to at least one second attribute of the first user while the first user uses the treatment apparatus to perform the treatment plan, wherein the instructions, when executed, cause the processing device to:

responsive to receiving the third data, determine whether at least one second attribute of the second user matches with at least one attribute of a third user, and whether at least one attribute of the occupational task associated with the second user matches with at least one attribute of an occupational task associated with the third user;

responsive to determining that at least one second attribute of the second user matches with at least one attribute of the third user, and that at least one attribute of the occupational task associated with the second user matches with at least one attribute of an occupational task associated with the third user, predict, via the artificial intelligence engine, a second estimate of when the second user performing the treatment plan would be capable of performing the occupational task associated with the second user.

40. The tangible, non-transitory computer-readable medium of clause 39, wherein the instructions, when executed, cause the processing device to:

transmit at least a portion of the third data, at least a portion of the treatment plan, and data pertaining to the second estimate to a computing device of a healthcare professional during a telemedicine session.

41. The tangible, non-transitory computer-readable medium of clause 39, wherein the third user is assigned to a second cohort based at least in part on the third data, wherein the instructions, when executed, cause the processing device to:

assign the second user to the second cohort.

42. The tangible, non-transitory computer-readable medium of clause 37, wherein the third data pertains to at least one second attribute of the first user while the first user uses the treatment apparatus to perform the treatment plan, wherein the instructions, when executed, cause the processing device to:

receive fourth data pertaining to a third user using the treatment apparatus to perform a second treatment plan;

responsive to receiving the fourth data, determine whether at least one second attribute of the second user matches with at least one attribute of the third user, and whether at least one attribute of the occupational task associated with the second user matches with at least one attribute of an occupational task associated with the third user;

responsive to determining that at least one second attribute of the second user matches with at least one attribute of the third user, and that at least one attribute of the occupational task associated with the second user matches with at least one attribute of an occupational task associated with the third user, predict, via the artificial intelligence engine, a second estimate of when the second user performing the second treatment plan would be capable of performing the occupational task associated with the second user.

43. The tangible, non-transitory computer-readable medium of clause 42, wherein the instructions, when executed, cause the processing device to:

transmit at least a portion of the fourth data, at least a portion of the second treatment plan, and the second estimate to a computing device of the healthcare professional during a telemedicine session; and

receive, from the computing device of a healthcare professional, a selection of the second treatment plan for the second user.

44. The tangible, non-transitory computer-readable medium of clause 43, wherein the third user is assigned to a second cohort based at least in part on the fourth data, wherein the instructions, when executed, cause the processing device to:

responsive to receiving the selection of the second treatment plan for the second user from the computing device of the healthcare professional, assign the second user to the second cohort.

45. The tangible, non-transitory computer-readable medium of clause 27, wherein the treatment apparatus used by the first user and the treatment apparatus used by the second user are the same.

46. The tangible, non-transitory computer-readable medium of clause 45, wherein the treatment apparatus used by the first user is the treatment apparatus used by the second user.

47. The tangible, non-transitory computer-readable medium of clause 35, wherein the data pertaining to the estimate comprises data pertaining to a prediction of an estimate of a cost of the first user becoming capable of performing the occupational task associated with the second user.

48. The tangible, non-transitory computer-readable medium of clause 27, wherein the artificial intelligence engine trains a machine learning model, using at least the first data, to compare in real-time the second data to a plurality of data and to predict the estimate.

49. The tangible, non-transitory computer-readable medium of clause 27, wherein the artificial intelligence engine trains the machine learning model, using at least the first data, to compare in real-time the second data to the plurality of data and to select at least one awardable incentive to offer to the second user for adhering to the treatment plan.

50. The tangible, non-transitory computer-readable medium of clause 27, wherein the instructions, when executed, cause the processing device to:

control, based on the treatment plan and while the second user uses the treatment apparatus, the treatment apparatus.

51. The tangible, non-transitory computer-readable medium of clause 50, wherein the instructions, when executed, cause the processing device to:

prior to controlling the treatment apparatus while the second user uses the treatment apparatus, provide to a computing device of a healthcare professional, during a telemedicine session, a recommendation pertaining to the treatment plan;

receive, from the computing device of the healthcare professional, at least a portion of the treatment plan; and

control, based on the at least a portion of the treatment plan and while the second user uses the treatment apparatus, the treatment apparatus.

52. The tangible, non-transitory computer-readable medium of clause 50, wherein the controlling is performed by a server distal from the treatment apparatus.

53. A system comprising:

a memory device storing instructions;

a processing device communicatively coupled to the memory device, the processing device executes the instructions to:

receive first data pertaining to a first user using a treatment apparatus to perform a treatment plan, wherein the first data comprises at least one attribute of the first user and at least one attribute of an occupational task associated with the first user;

receive second data pertaining to a second user, wherein the second data comprises at least one attribute of the second user and at least one attribute of an occupational task associated with the second user;

determine whether at least one attribute of the second user matches with at least one attribute of the first user, and whether at least one attribute of the occupational task associated with the second user matches with at least one attribute of the occupational task associated with the first user; and

responsive to determining that at least one attribute of the second user matches with at least one attribute of the first user and that at least one attribute of the occupational task associated with the second user matches with at least one attribute of the occupational task associated with the first user, predict, via an artificial intelligence engine, an estimate of when the second user performing the treatment plan would be capable of performing the occupational task associated with the second user.

54. The system of clause 53, wherein the at least one attribute of the first user and of the second user comprises personal information, performance information, measurement information, incentive information, or some combination thereof.

55. The system of clause 54, wherein:

the personal information comprises an age, a weight, a gender, a height, a body mass index, a medical condition, a familial medication history, an injury, a medical procedure, or some combination thereof,

the performance information comprises an elapsed time of using the treatment apparatus, an amount of force exerted on a portion of the treatment apparatus, a range of motion achieved on the treatment apparatus, a movement speed of a portion of the treatment apparatus, an indication of a plurality of pain levels using the treatment apparatus, or some combination thereof,

the measurement information comprises a vital sign, a respiration rate, a heartrate, a temperature, or some combination thereof, and

the incentive information comprises information about an awardable product, object, activity, accolade, title, discount, coupon, or some combination thereof.

56. The system of clause 53, wherein the at least one attribute of an occupational task of the first user and of the second user comprises a stamina requirement, a strength requirement, a flexibility requirement, a pliability requirement, a range of motion requirement, a sustained attention requirement, a speech requirement, a height requirement, a weight requirement, a limb use requirement, or some combination thereof.

57. The system of clause 53, wherein to predict an estimate of when the second user performing the treatment plan would be capable of performing the occupational task associated with the second user, the processing device executes the instructions to:

predict an estimate of when a readiness score of the second user performing the treatment plan would satisfy a readiness score threshold for the occupational task associated with the second user.

58. The system of clause 57, wherein:

the readiness score is based on at least one capability score of the second user; and

the readiness score threshold is based on at least one capability score threshold for the occupational task associated with the second user.

59. The system of clause 58, wherein the at least one capability score is selected from the group consisting of: a stamina score, a strength score, a flexibility score, a pliability score, a range of motion score, a sustained attention score, a speech score, a height score, a weight score, a limb use score, or some combination thereof; and

the at least one capability score threshold is selected from the group consisting of: a stamina score threshold, a strength score threshold, a flexibility score threshold, a pliability score threshold, a range of motion score threshold, a sustained attention score threshold, a speech score threshold, a height score threshold, a weight score threshold, a limb use score threshold, or some combination thereof.

60. The system of clause 58, wherein:

the readiness score is a weighted average of a plurality of capability scores of the second user; and

the readiness score threshold is a weighted average of a plurality of capability score thresholds for the occupational task associated with the second user.

61. The system of clause 53, wherein the processing device executes the instructions to:

transmit at least a portion of the second data, at least a portion of the treatment plan, and data pertaining to the estimate to a computing device of a healthcare professional during a telemedicine session; and

receive, from the computing device of a healthcare professional, a selection of the treatment plan for the second user.

62. The system of clause 61, wherein the first user is assigned to a first cohort based at least in part on the first data, wherein the processing device executes the instructions to:

responsive to receiving the selection of the treatment plan for the second user from the computing device of the healthcare professional, assign the second user to the first cohort.

63. The system of clause 62, wherein the processing device executes the instructions to:

receive, from the treatment apparatus, third data pertaining to use of the treatment apparatus by the second user performing the treatment plan; and

provide, to a computing device of the healthcare professional, a report pertaining to the third data.

64. The system of clause 63, wherein the processing device executes the instructions to:

responsive to receiving the third data, predict, via the artificial intelligence engine, a second estimate of when the second user performing the treatment plan would be capable of performing the occupational task associated with the second user; and

provide, to the computing device of the healthcare professional, a report pertaining to the second estimate.

65. The system of clause 63, wherein the third data pertains to at least one second attribute of the first user while the first user uses the treatment apparatus to perform the treatment plan, wherein the processing device executes the instructions to:

responsive to receiving the third data, determine whether at least one second attribute of the second user matches with at least one attribute of a third user, and whether at least one attribute of the occupational task associated with the second user matches with at least one attribute of an occupational task associated with the third user;

responsive to determining that at least one second attribute of the second user matches with at least one attribute of the third user, and that at least one attribute of the occupational task associated with the second user matches with at least one attribute of an occupational task associated with the third user, predict, via the artificial intelligence engine, a second estimate of when the second user performing the treatment plan would be capable of performing the occupational task associated with the second user.

66. The system of clause 65, wherein the processing device executes the instructions to:

transmit at least a portion of the third data, at least a portion of the treatment plan, and data pertaining to the second estimate to a computing device of a healthcare professional during a telemedicine session.

67. The system of clause 65, wherein the third user is assigned to a second cohort based at least in part on the third data, wherein the processing device executes the instructions to:

assign the second user to the second cohort.

68. The system of clause 63, wherein the third data pertains to at least one second attribute of the first user while the first user uses the treatment apparatus to perform the treatment plan, wherein the processing device executes the instructions to:

receive fourth data pertaining to a third user using the treatment apparatus to perform a second treatment plan;

responsive to receiving the fourth data, determine whether at least one second attribute of the second user matches with at least one attribute of the third user, and whether at least one attribute of the occupational task associated with the second user matches with at least one attribute of an occupational task associated with the third user;

responsive to determining that at least one second attribute of the second user matches with at least one attribute of the third user, and that at least one attribute of the occupational task associated with the second user matches with at least one attribute of an occupational task associated with the third user, predict, via the artificial intelligence engine, a second estimate of when the second user performing the second treatment plan would be capable of performing the occupational task associated with the second user.

69. The system of clause 68, wherein the processing device executes the instructions to:

transmit at least a portion of the fourth data, at least a portion of the second treatment plan, and the second estimate to a computing device of the healthcare professional during a telemedicine session; and

receive, from the computing device of a healthcare professional, a selection of the second treatment plan for the second user.

70. The system of clause 69, wherein the third user is assigned to a second cohort based at least in part on the fourth data, wherein the processing device executes the instructions to:

responsive to receiving the selection of the second treatment plan for the second user from the computing device of the healthcare professional, assign the second user to the second cohort.

71. The system of clause 53, wherein the treatment apparatus used by the first user and the treatment apparatus used by the second user are the same.

72. The system of clause 71, wherein the treatment apparatus used by the first user is the treatment apparatus used by the second user.

73. The system of clause 61, wherein the data pertaining to the estimate comprises data pertaining to a prediction of an estimate of a cost of the first user becoming capable of performing the occupational task associated with the second user.

74. The system of clause 53, wherein the artificial intelligence engine trains a machine learning model, using at least the first data, to compare in real-time the second data to a plurality of data and to predict the estimate.

75. The system of clause 53, wherein the artificial intelligence engine trains the machine learning model, using at least the first data, to compare in real-time the second data to the plurality of data and to select at least one awardable incentive to offer to the second user for adhering to the treatment plan.

76. The system of clause 53, wherein the processing device executes the instructions to:

control, based on the treatment plan and while the second user uses the treatment apparatus, the treatment apparatus.

77. The system of clause 76, wherein the processing device executes the instructions to:

prior to controlling the treatment apparatus while the second user uses the treatment apparatus, provide to a computing device of a healthcare professional, during a telemedicine session, a recommendation pertaining to the treatment plan;

receive, from the computing device of the healthcare professional, at least a portion of the treatment plan; and

control, based on the at least a portion of the treatment plan and while the second user uses the treatment apparatus, the treatment apparatus.

78. The system of clause 76, wherein the controlling is performed by a server distal from the treatment apparatus.

The above discussion is meant to be illustrative of the principles and various embodiments of the present disclosure. Numerous variations and modifications will become apparent to those skilled in the art once the above disclosure is fully appreciated. It is intended that the following clauses be interpreted to embrace all such variations and modifications.

The various aspects, embodiments, implementations, or features of the described embodiments can be used separately or in any combination. The embodiments disclosed herein are modular in nature and can be used in conjunction with or coupled to other embodiments.

Consistent with the above disclosure, the examples of assemblies enumerated in the following clauses are specifically contemplated and are intended as a non-limiting set of examples.

What is claimed is:

1. A method comprising:
receiving first data pertaining to a first user using a treatment apparatus to perform a treatment plan, wherein the first data comprises at least one attribute of the first user and at least one attribute of an occupational task associated with the first user;
receiving second data pertaining to a second user, wherein the second data comprises at least one attribute of the second user and at least one attribute of an occupational task associated with the second user;
determining whether at least one attribute of the second user matches with at least one attribute of the first user, and whether at least one attribute of the occupational task associated with the second user matches with at least one attribute of the occupational task associated with the first user; and
responsive to determining that at least one attribute of the second user matches with at least one attribute of the first user and that at least one attribute of the occupational task associated with the second user matches with at least one attribute of the occupational task associated with the first user, predicting, via an artificial intelligence engine, an estimate of when the second user performing the treatment plan would be capable of performing the occupational task associated with the second user.

2. The method of claim 1, wherein the at least one attribute of the first user and of the second user comprises personal information, performance information, measurement information, incentive information, or some combination thereof.

3. The method of claim 2, wherein:
the personal information comprises an age, a weight, a gender, a height, a body mass index, a medical condition, a familial medication history, an injury, a medical procedure, or some combination thereof,
the performance information comprises an elapsed time of using the treatment apparatus, an amount of force exerted on a portion of the treatment apparatus, a range of motion achieved on the treatment apparatus, a movement speed of a portion of the treatment apparatus, an indication of a plurality of pain levels using the treatment apparatus, or some combination thereof,
the measurement information comprises a vital sign, a respiration rate, a heartrate, a temperature, or some combination thereof, and
the incentive information comprises information about an awardable product, object, activity, accolade, title, discount, coupon, or some combination thereof.

4. The method of claim 1, wherein the at least one attribute of an occupational task of the first user and of the second user comprises a stamina requirement, a strength requirement, a flexibility requirement, a pliability requirement, a range of motion requirement, a sustained attention requirement, a speech requirement, a height requirement, a weight requirement, a limb use requirement, or some combination thereof.

5. The method of claim 1, wherein the predicting comprises:
predicting an estimate of when a readiness score of the second user performing the treatment plan would satisfy a readiness score threshold for the occupational task associated with the second user.

6. The method of claim 5, wherein:
the readiness score is based on at least one capability score of the second user; and
the readiness score threshold is based on at least one capability score threshold for the occupational task associated with the second user.

7. The method of claim 6, wherein the at least one capability score is selected from the group consisting of: a stamina score, a strength score, a flexibility score, a pliability score, a range of motion score, a sustained attention score, a speech score, a height score, a weight score, a limb use score, or some combination thereof; and
the at least one capability score threshold is selected from the group consisting of: a stamina score threshold, a strength score threshold, a flexibility score threshold, a pliability score threshold, a range of motion score threshold, a sustained attention score threshold, a speech score threshold, a height score threshold, a weight score threshold, a limb use score threshold, or some combination thereof.

8. The method of claim 6, wherein:
the readiness score is a weighted average of a plurality of capability scores of the second user; and
the readiness score threshold is a weighted average of a plurality of capability score thresholds for the occupational task associated with the second user.

9. The method of claim 1, further comprising:
transmitting at least a portion of the second data, at least a portion of the treatment plan, and data pertaining to the estimate to a computing device of a healthcare professional during a telemedicine session; and
receiving, from the computing device of a healthcare professional, a selection of the treatment plan for the second user.

10. The method of claim 9, wherein the first user is assigned to a first cohort based at least in part on the first data, the method comprising:
responsive to receiving the selection of the treatment plan for the second user from the computing device of the healthcare professional, assigning the second user to the first cohort.

11. The method of claim 10, comprising:
receiving, from the treatment apparatus, third data pertaining to use of the treatment apparatus by the second user performing the treatment plan; and
providing, to a computing device of the healthcare professional, a report pertaining to the third data.

12. The method of claim 11, comprising:
responsive to receiving the third data, predicting, via the artificial intelligence engine, a second estimate of when the second user performing the treatment plan would be capable of performing the occupational task associated with the second user; and
providing, to the computing device of the healthcare professional, a report pertaining to the second estimate.

13. The method of claim 11, wherein the third data pertains to at least one second attribute of the first user while the first user uses the treatment apparatus to perform the treatment plan, the method comprising:
responsive to receiving the third data, determining whether at least one second attribute of the second user matches with at least one attribute of a third user, and whether at least one attribute of the occupational task associated with the second user matches with at least one attribute of an occupational task associated with the third user;
responsive to determining that at least one second attribute of the second user matches with at least one attribute of the third user, and that at least one attribute of the occupational task associated with the second user matches with at least one attribute of an occupational task associated with the third user, predicting, via the artificial intelligence engine, a second estimate of when the second user performing the treatment plan would be capable of performing the occupational task associated with the second user.

14. The method of claim 13, further comprising:
transmitting at least a portion of the third data, at least a portion of the treatment plan, and data pertaining to the second estimate to a computing device of a healthcare professional during a telemedicine session.

15. The method of claim 13, wherein the third user is assigned to a second cohort based at least in part on the third data, the method comprising:
assigning the second user to the second cohort.

16. The method of claim 11, wherein the third data pertains to at least one second attribute of the first user while the first user uses the treatment apparatus to perform the treatment plan, the method comprising:
receiving fourth data pertaining to a third user using the treatment apparatus to perform a second treatment plan;
responsive to receiving the fourth data, determining whether at least one second attribute of the second user matches with at least one attribute of the third user, and whether at least one attribute of the occupational task associated with the second user matches with at least one attribute of an occupational task associated with the third user;
responsive to determining that at least one second attribute of the second user matches with at least one attribute of the third user, and that at least one attribute of the occupational task associated with the second user matches with at least one attribute of an occupational task associated with the third user, predicting, via the artificial intelligence engine, a second estimate of when the second user performing the second treatment plan would be capable of performing the occupational task associated with the second user.

17. The method of claim 16, further comprising:
transmitting at least a portion of the fourth data, at least a portion of the second treatment plan, and the second estimate to a computing device of the healthcare professional during a telemedicine session; and
receiving, from the computing device of a healthcare professional, a selection of the second treatment plan for the second user.

18. The method of claim 17, wherein the third user is assigned to a second cohort based at least in part on the fourth data, the method comprising:
responsive to receiving the selection of the second treatment plan for the second user from the computing device of the healthcare professional, assigning the second user to the second cohort.

19. A tangible, non-transitory computer-readable medium storing instructions that, when executed, cause a processing device to:
receive first data pertaining to a first user using a treatment apparatus to perform a treatment plan, wherein the first data comprises at least one attribute of the first user and at least one attribute of an occupational task associated with the first user;
receive second data pertaining to a second user, wherein the second data comprises at least one attribute of the second user and at least one attribute of an occupational task associated with the second user;
determine whether at least one attribute of the second user matches with at least one attribute of the first user, and whether at least one attribute of the occupational task associated with the second user matches with at least one attribute of the occupational task associated with the first user; and
responsive to determining that at least one attribute of the second user matches with at least one attribute of the first user and that at least one attribute of the occupational task associated with the second user matches with at least one attribute of the occupational task associated with the first user, predict, via an artificial intelligence engine, an estimate of when the second user performing the treatment plan would be capable of performing the occupational task associated with the second user.

20. A system comprising:
a memory device storing instructions;
a processing device communicatively coupled to the memory device, the processing device executes the instructions to:
receive first data pertaining to a first user using a treatment apparatus to perform a treatment plan, wherein the first data comprises at least one attribute of the first user and at least one attribute of an occupational task associated with the first user;
receive second data pertaining to a second user, wherein the second data comprises at least one attribute of the second user and at least one attribute of an occupational task associated with the second user;
determine whether at least one attribute of the second user matches with at least one attribute of the first user, and whether at least one attribute of the occupational task associated with the second user matches with at least one attribute of the occupational task associated with the first user; and
responsive to determining that at least one attribute of the second user matches with at least one attribute of the first user and that at least one attribute of the occupational task associated with the second user matches with at least one attribute of the occupational task associated with the first user, predict, via an artificial intelligence engine, an estimate of when the second user performing the treatment plan would be capable of performing the occupational task associated with the second user.
